(19) United States
(12) Patent Application Publication
(10) Pub. No.: US 2005/0255498 A1
(43) Pub. Date: Nov. 17, 2005

(54) APOC1 genetic markers associated with age of onset of Alzheimer's Disease

(75) Inventors: Jeroen Aerssens, Nieuwrode (BE); Maria Athanasiou, Derby, CT (US); Carlos Brain, Somerville, MA (US); Nadine Cohen, Warren, NJ (US); Bradley Dain, Hamden, CT (US); R. Rex Denton, Madison, CT (US); Richard S. Judson, Guilford, CT (US); Vural Ozdemir, Long Beach, CA (US); Carol R. Reed, Bethany, CT (US)

Correspondence Address: STERNE, KESSLER, GOLDSTEIN & FOX PLLC, 1100 NEW YORK AVENUE, N.W., WASHINGTON, DC, 20005, US

(73) Assignee: Genaissance Pharmaceuticals

(21) Appl. No.: 11/035,105

(22) Filed: Jan. 14, 2005

Related U.S. Application Data
(60) Provisional application No. 60/538,606, filed on Jan. 22, 2004.

(51) Int. Cl. C12Q 1/68

(52) U.S. Cl. 435/6

(57) ABSTRACT

Haplotypes in the APOC1 gene associated with age of onset of Alzheimer's Disease are disclosed. Compositions and methods for detecting and using these APOC1 haplotypes in a variety of clinical applications are disclosed. Such applications include articles of manufacture comprising compounds effective in delaying the age of onset of AD in individuals at risk for developing AD and having one of these APOC1 haplotypes, methods and kits for predicting the age of onset of AD in an individual at risk for developing AD based upon his/her haplotype profile, and methods for delaying the age of onset of AD in individuals at risk for developing AD based upon their haplotype profile.

### FIELD OF THE INVENTION

This invention relates to the fields of genomics and pharmacogenomics. More specifically, this invention relates to variants of the gene encoding apolipoprotein C-1 (APOC1) and their association with age of onset of Alzheimer's Disease.

### BACKGROUND OF THE INVENTION

Alzheimer's Disease (hereinafter “AD”) is a fatal degenerative disorder of the central nervous system that is characterized by profound memory impairment, emotional disturbance, and in late stages, personality changes (Bartolucci et al., Proteins 42:182-91 (2001)). Scientists generally distinguish between sporadic and familial AD. Sporadic AD, a late-onset form of the disease, is the most common form of AD, and generally only occurs in people who are at least 65. Familial AD, an early-onset form of the disease, and accounting for only about 5% of all AD cases, generally affects people between the ages of 30 and 65. The average worldwide risk of developing any type of AD is about 5% by age 65, 10 to 15% by age 75, and 20 to 40% by age 85. While the cause of sporadic AD is unknown, genetic factors are believed to be involved, as evidenced by an increased risk of AD in individuals who have a family history of AD (Devi et al., Arch. Neurol. 57:28-9 (2000)) or who have one or more of several specific polymorphisms that have been correlated with increased risk for AD. Known genetic polymorphisms that are risk factors for developing AD include the apolipoprotein E (APOE) ε4 allele (U.S. Pat. No. 5,508,167); the al -antichymotrypsin (ACT) A allele (U.S. Pat. No. 5,773,220), and the interleukin-1 (IL-1) A 2,2 and IL-1B 2,2 genotypes (U.S. Pat. No. 6,225,069 B1).

Another recently recognized risk factor for developing AD is a diagnosis of mild or minimal cognitive impairment (MCI), which is a condition characterized by subtle cognitive deficits not severe enough to be classified as true dementia, but in many patients, and perhaps all, represents an early stage of AD (see, e.g., Chertkow, Curr. Opin. Neurol. 15:401-7 (2002); Morris et al., Arch. Neurol. 58:397-405 (2001); Almkvist et al., J. Neural Transm. Suppl. 54:21-9 (1998)). It has been suggested that if drug therapy were started when symptoms of reduced cognitive function first appear, even before a clinical diagnosis of AD, it is possible that progression to AD could be delayed or prevented (Morris et al., supra). Several multicenter trials of various pharmacological agents are underway to test this hypothesis (Petersen et al., Neurology 56:1133-42 (2001)).

A positive outcome of these trials may have a significant societal impact. In 1998, the annual cost in the United States for the care of patients with AD was about $40,000 per patient and it is estimated that there will be 14 million AD patients in the United States by the year 2050 (Petersen et al., supra). Thus, a pharmacological treatment that delays the progression of AD by as little as a year could result in huge cost savings and provide afflicted individuals with additional time to plan for their future while their decision-making capacity is only minimally affected.

This potential for pharmacological intervention to delay the onset or progression of AD will place increasing pressure on health care professionals to diagnose whether an individual has MCI or early stage AD. However, there is controversy surrounding the characterization and definition of MCI, and early symptoms of AD are frequently mistakenly attributed to the normal aging process (Morris et al., supra; Petersen et al., supra). Thus, a need exists for improved methods of diagnosing MCI and early stage AD.

A gene that may be involved in the age of onset of AD is apolipoprotein C-1 (APOC1). The gene encoding APOC1 consists of four exons (Lauer et al., J. Biol. Chem. 263(15):7277-86 (1988)) and has been mapped to chromosomal band 19q13.2 (Trask et al., Genomics 15:133-45 (1993)). APOC1 forms a gene cluster with the gene encoding apolipoprotein E (APOE) (Smit et al., Hum. Genet. 78:90-3 (1988)). APOE is expressed mainly in astrocytes within the central nervous system (Poierier et al., Mol. Brain Res. 11:97-106 (1991)), and is involved in redistributing cholesterol and phospholipid during membrane remodeling associated with synaptic plasticity (Poirier et al., Neuroscience 55:81-90 (1993)). The gene encoding APOE has three alleles, ε2, ε3, and ε4. In 1993, Corder et al. discovered that the APOE ε4 allele is associated, in a dose-dependent manner, with the age of onset of AD (Science 261:921-3).

Because of the possible involvement of APOC1 in the cognitive deficits observed in MCI and early stages of AD, and the need for additional ways to identify people with these conditions, it would be useful to assess the degree of variation in the APOC1 gene in patients with AD and to determine if any variants of this gene are associated with the age of AD onset.

### SUMMARY OF THE INVENTION

Accordingly, the inventors herein have discovered a set of haplotypes in the APOC1 gene that are associated with the age of onset of AD. The inventors have also discovered that the copy number of each of these APOC1 haplotypes affects the age of onset of AD. Testing for the presence or absence, and copy number, of these haplotypes is useful for predicting the age at which individuals who are at increased risk for AD are likely to develop AD and to help confirm a diagnosis of MCI or AD. Such knowledge will help individuals with MCI or AD, as well as their physicians and families, make therapy and lifestyle decisions. In addition, the correlation of certain APOC1 haplotypes with age of AD onset indicates that variation in the APOC1 gene should be considered in the development and clinical trials of drugs for treating MCI, AD and other neurodegenerative disorders. This correlation also provides a basis for pursuing APOC1 as a target for drugs designed to treat cognitive disorders such as MCI, AD and other neurological diseases or conditions. The APOC1 haplotypes are shown in Table 1 below.

TABLE 1
APOC1 Haplotypes Having Association with
Age of Onset of Alzheimer's Disease

| | Polymorphic Site (PS)¹ | | | | | | | | | |
|---|---|---|---|---|---|---|---|---|---|---|
| Haplotype | 1 | 2 | 3² | 4 | 5 | 6 | 7 | 8 | 9 | 10 |

| | | | | | | | | |
|---|---|---|---|---|---|---|---|---|
| (1) | | | del | | | C | | |
| (2) | | | del | A | | C | G | |
| (3) | A | | del | | | C | G | |
| (4) | | | del | | C | C | G | |
| (5) | | G | del | | | C | G | |
| (6) | | | del | | | C | G | C |
| (7) | | G | del | A | | C | | |
| (8) | | | del | A | | C | | |
| (9) | A | | del | | | C | | |
| (10) | | G | del | | | C | | |
| (11) | | | del | | C | C | | |
| (12) | | | del | | | C | | C |
| (13) | A | | del | A | | C | | |
| (14) | | | del | A | C | C | | |
| (15) | A | | del | | C | C | | |
| (16) | A | G | del | | | C | | |
| (17) | | G | del | | C | C | | |
| (18) | | | del | A | | C | | C |
| (19) | A | | del | | | C | | C |
| (20) | | | del | | | C | G | |
| (21) | | | del | | C | C | | C |
| (22) | | G | del | | | C | | C |
| (23) | | | del | | | | | |
| (24) | | | del | A | C | | G | |
| (25) | | | del | A | | | | C |
| (26) | | G | del | | | | G | C |
| (27) | A | | del | | C | | | C |
| (28) | A | | del | A | | | | |
| (29) | A | G | del | | | | G | |
| (30) | | | del | | C | | G | |
| (31) | | | del | | | | | C |
| (32) | | G | del | A | | | G | |
| (33) | A | | del | | | | | |
| (34) | | | del | A | C | | | C |
| (35) | A | | del | A | C | | | |
| (36) | A | G | del | | | | | C |
| (37) | | | del | A | | | | |
| (38) | | | del | | C | | | C |
| (39) | | G | del | | | | G | |
| (40) | A | | del | | C | | | |
| (41) | | G | del | A | | | | C |
| (42) | A | G | del | A | | | | |
| (43) | | | del | A | C | | | |
| (44) | A | | del | | | | G | C |
| (45) | | G | del | | C | | G | |
| (46) | | G | del | | | | | C |
| (47) | A | G | del | | | | | |
| (48) | | | del | | C | | | |
| (49) | | | del | A | | | G | C |
| (50) | A | | del | A | | | G | |
| (51) | | G | del | A | | | | |
| (52) | | G | del | | C | | | C |
| (53) | | | del | | | | G | C |
| (54) | A | G | del | | C | | | |
| (55) | A | | del | | | | G | |
| (56) | | G | del | | | | | |
| (57) | | G | del | A | C | | | |
| (58) | A | | del | A | | | | C |
| (59) | | | del | | C | | G | C |
| (60) | | | del | A | | | G | |
| (61) | A | | del | | C | | G | |
| (62) | A | | del | | | | | C |
| (63) | | G | del | | C | | | |
| (64) | | | del | | | | G | |
| (65) | | | — | | | | | |
| (66) | | | — | | | C | | |
| (67) | A | | — | | | | | |
| (68) | A | | — | | | C | | |

¹The absence of a PS entry for a haplotype indicates that the PS is not part of the marker.
²“del” = deletion; “—” = no deletion.

If an individual has (a) one copy or two copies of any of haplotypes (1)-(64) in Table 1, or (b) zero copies or one copy of any of haplotypes (65)-(68) in Table 1, that individual is defined as having an “age of onset marker I” and is more likely to have a later age of onset of AD than an individual having (a) zero copies of any of haplotypes (1)-(64) in Table 1, or (b) two copies of any of haplotypes (65)-(68) in Table 1, such individual being defined as having an “age of onset marker II.” Information about the composition of each of haplotypes (1)-(68) in Table 1, namely the location in the APOC1 gene of each of the polymorphic sites (PSs), and the identity of the reference and variant allele at each PS, can be found in Table 2, shown below.

TABLE 2
Polymorphic Sites Identified in the APOC1 Gene of
Caucasian Individuals with Alzheimer's Disease

| | | | Position in | | |
|---|---|---|---|---|---|
| PS | | | FIG. 1/ | Reference | Variant |
| Number | Poly ID¹ | Location | SEQ ID NO:1 | Allele | Allele |
| 1 | 613389718 | promoter | 106 | A | T |
| 2 | 20321030 | promoter | 110 | G | C |
| 3 | 614132292 | promoter | 172 | — | CGTT |
| | | | | | deletion |
| 4 | 613258552 | promoter | 381 | G | C |
| 5 | 613238812 | promoter | 536 | A | G |
| 6 | 613238814 | promoter | 555 | C | G |
| 7 | 3792343 | exon 1 | 731 | C | G |
| 8 | 613238817 | intron 1 | 773 | G | A |
| 9 | 3792345 | intron 1 | 787 | G | A |
| 10 | 613243388 | intron 1 | 827 | C | G |

¹The Poly ID is a unique identifier assigned to the indicated PS by Genaissance Pharmaceuticals, Inc., New Haven, CT.

In addition, as described in more detail below, the inventors believe that additional haplotypes may readily be identified based on linkage disequilibrium between any of the above APOC1 haplotypes and another haplotype located in the APOC1 gene or another gene, or between an allele at one or more of the PSs in the above haplotypes and an allele at another PS located in the APOC1 gene or another gene. In particular, such haplotypes include haplotypes that are in linkage disequilibrium with any of haplotypes (1)-(68) in Table 1, hereinafter referred to as “linked haplotypes,” as well as “substitute haplotypes” for any of haplotypes (1)-(68) in which one or more of the polymorphic sites (PSs) in the original haplotype is substituted with another PS, wherein the allele at the substituted PS is in linkage disequilibrium with the allele at the substituting PS.

In one aspect, the invention provides methods and kits for determining whether an individual has an age of onset marker I or an age of onset marker II. In one embodiment, a method is provided for determining whether an individual has an age of onset marker I or an age of onset marker II comprising determining whether the individual has (a) one copy or two copies, or zero copies of any of (i) haplotypes (1)-(64) in Table 1, (ii) a linked haplotype for any of haplotypes (1)-(64) in Table 1, and (iii) a substitute haplotype for any of haplotypes (1)-(64) in Table 1, or (b) zero copies or one copy, or two copies of any of (i) haplotypes (65)-(68) in Table 1, (ii) a linked haplotype for any of haplotypes (65)-(68) in Table 1, and (iii) a substitute haplotype for any of haplotypes (65)-(68) in Table 1.

In another embodiment of the invention, a method is provided for assigning an individual to a first or second age of onset marker group comprising determining whether the individual has (a) one copy or two copies, or zero copies of any of (i) haplotypes (1)-(64) in Table 1, (ii) a linked haplotype for any of haplotypes (1)-(64) in Table 1, and (iii) a substitute haplotype for any of haplotypes (1)-(64) in Table 1, or (b) zero copies or one copy, or two copies of any of (i) haplotypes (65)-(68), (ii) a linked haplotype for any of haplotypes (65)-(68) in Table 1, and (iii) a substitute haplotype for any of haplotypes (65)-(68) in Table 1; and assigning the individual to an age of onset marker group based on the copy number of that haplotype. The individual is assigned to the first age of onset marker group if the individual has (a) one copy or two copies of any of (i) haplotypes (1)-(64) in Table 1, (ii) a linked haplotype for any of haplotypes (1)-(64) in Table 1, and (iii) a substitute haplotype for any of haplotypes (l)-(64) in Table 1, or (b) zero copies or one copy of any of (i) haplotypes (65)-(68), (ii) a linked haplotype for any of haplotypes (65)-(68) in Table 1, and (iii) a substitute haplotype for any of haplotypes (65)-(68) in Table 1, and is assigned to the second age of onset marker group if the individual has (a) zero copies of any of (i) haplotypes (1)-(64) in Table 1, (ii) a linked haplotype for any of haplotypes (1)-(64) in Table 1, and (iii) a substitute haplotype for any of haplotypes (1)-(64) in Table 1, or (b) two copies of any of (i) haplotypes (65)-(68), (ii) a linked haplotype for any of haplotypes (65)-(68) in Table 1, and (iii) a substitute haplotype for any of haplotypes (65)-(68) in Table 1.

One embodiment of a kit for determining whether an individual has an age of onset marker I or an age of onset marker II comprises a set of oligonucleotides designed for identifying at least one of the alleles present at each PS in a set of one or more PSs. The set of one or more PSs comprises the set of one or more PSs for any of the haplotypes in Table 1, the set of one or more PSs for a linked haplotype for any of the haplotypes in Table 1, or the set of one or more PSs for a substitute haplotype for any of the haplotypes in Table 1. In a further embodiment, the kit comprises a manual with instructions for performing one or more reactions on a human nucleic acid sample to identify the allele(s) present in the individual at each PS in the set and determining if the individual has an age of onset marker I or an age of onset marker II based on the identified allele(s).

The invention further provides a method for delaying the onset of AD in an individual at risk for developing AD. The method comprises determining whether the individual has an age of onset marker I or an age of onset marker II and making a treatment decision for the individual based on the results of the determining step. If the individual is determined to have an age of onset marker I, then the treatment decision is to prescribe to the individual a compound effective in delaying the onset of AD, wherein the compound is prescribed to the individual at an age that is below that of the lower confidence interval of the least square mean of age of onset for the age of onset marker I. If the individual is determined to have an age of onset marker II, then the treatment decision is to prescribe to the individual a compound effective in delaying the onset of AD, wherein the compound is prescribed to the individual at an age that is below that of the lower confidence interval of the least square mean of age of onset for the age of onset marker II. According to Table 7 below, the lower confidence interval of the least square mean of age of onset for an age of onset marker I is 72.1, and the lower confidence interval of the least square mean of age of onset for an age of onset marker II ranges from 68.3 to 68.4.

In yet another embodiment, the invention provides a method for predicting an individual's age of onset of AD. The method comprises determining whether the individual has an age of onset marker I or an age of onset marker II and making an prediction based on the results of the determining step. According to Table 7 below, if the individual is determined to have an age of onset marker I, then the prediction is that the individual's age of onset of AD will be between 72.1 and 73.9, and if the individual is determined to have an age of onset marker II, then the prediction is that the individual's age of onset of AD will be between 68.3 and 72.2.

In other aspects, the invention provides (i) a method for seeking regulatory approval for marketing a pharmaceutical formulation comprising, as at least one active ingredient, a compound effective in delaying the onset of AD, to a population at risk for developing AD, wherein the population is partially or wholly defined by having an age of onset marker I or an age of onset marker II, (ii) an article of manufacture comprising the pharmaceutical formulation, (iii) a method for manufacturing a drug product comprising the pharmaceutical formulation, and (iv) a method for marketing the drug product.

The method for seeking regulatory approval comprises conducting at least one clinical trial which comprises administering the pharmaceutical formulation to first and second groups of individuals at risk for developing AD, and administering a placebo to third and fourth groups of individuals at risk for developing AD, wherein each individual in the first and third groups has an age of onset marker I, and each individual in the second and fourth groups has an age of onset marker II, demonstrating that the first group exhibits a later onset of AD than the third group, and demonstrating that the second group exhibits a later onset of AD than the fourth group, and filing with a regulatory agency an application for marketing approval of the pharmaceutical formulation with a label stating that the pharmaceutical formulation is indicated for delaying the onset of AD in a population at risk for developing AD. In preferred embodiments, the regulatory agency is the United States Food and Drug Administration (FDA) or the European Agency for the Evaluation of Medicinal Products (EMEA), or a future equivalent of these agencies.

In one embodiment, the article of manufacture comprises the pharmaceutical formulation and at least one indicium identifying a population for whom the pharmaceutical formulation is indicated, wherein the identified population is at risk for developing AD and is partially or wholly defined by having an age of onset marker I or an age of onset marker II, wherein a trial population of individuals having an age of onset marker I exhibit a later age of onset of AD than a trial population having an age of onset marker II. Another embodiment of the article of manufacture comprises packaging material and the pharmaceutical formulation contained within the packaging material, wherein the packaging material comprises a label approved by a regulatory agency for the pharmaceutical formulation, wherein the label states that the pharmaceutical formulation is indicated for a population at risk for developing AD that is partially or wholly defined by having an age of onset marker I or an age of onset marker II, and preferably further stating that a trial population of individuals having an age of onset marker I exhibit a later age of onset of AD than a trial population of individuals having an age of onset marker II. Preferably, the pharmaceutical formulation comprises, as at least one active ingredient, a compound effective in delaying the onset of AD.

The method for manufacturing the drug product comprises combining in a package a pharmaceutical formulation comprising, as at least one active ingredient, a compound effective in delaying the onset of AD, and a label which states that the drug product is indicated for a population at risk for developing AD, wherein the population is partially or wholly defined by having an age of onset marker I or an age of onset marker II, wherein those members of the population having an age of onset marker I exhibit a later age of onset of AD than those members of the population having an age of onset marker II.

The method for marketing the drug product comprises promoting to a target audience the use of the drug product for treating individuals who belong to the defined population.

### BRIEF DESCRIPTION OF THE FIGURES

FIG. 1A-C illustrates a reference sequence for the APOC1 gene (contiguous lines; SEQ ID NO:1), with the start and stop positions of each region of coding sequence indicated with a bracket ([ or ]) and the numerical position below the sequence and the polymorphic site(s) and polymorphism(s) identified in the patient cohort indicated by the variant nucleotide positioned below the polymorphic site in the sequence.

### DEFINITIONS

In the context of this disclosure, the terms below shall be defined as follows unless otherwise indicated:
Allele—A particular form of a genetic locus, distinguished from other forms by its particular nucleotide sequence, or one of the alternative polymorphisms found at a polymorphic site.
Gene—A segment of DNA that contains the coding sequence for a protein, wherein the segment may include promoters, exons, introns, and other untranslated regions that control expression.
Genotype—An unphased 5′ to 3′ sequence of nucleotide pair(s) found at a set of one or more polymorphic sites in a locus on a pair of homologous chromosomes in an individual. As used herein, genotype includes a full-genotype and/or a sub-genotype as described below.
Genotyping—A process for determining a genotype of an individual.
Haplotype—A 5′ to 3′ sequence of nucleotides found at a set of one or more polymorphic sites in a locus on a single chromosome from a single individual.
Haplotype pair—The two haplotypes found for a locus in a single individual.
Haplotyping—A process for determining one or more haplotypes in an individual and includes use of family pedigrees, molecular techniques and/or statistical inference.
Haplotype data—Information concerning one or more of the following for a specific gene: a listing of the haplotype pairs in an individual or in each individual in a population; a listing of the different haplotypes in a population; frequency of each haplotype in that or other populations, and any known associations between one or more haplotypes and a trait.
Isolated—As applied to a biological molecule such as RNA, DNA, oligonucleotide, or protein, isolated means the molecule is substantially free of other biological molecules such as nucleic acids, proteins, lipids, carbohydrates, or other material such as cellular debris and growth media. Generally, the term “isolated” is not intended to refer to a complete absence of such material or to absence of water, buffers, or salts, unless they are present in amounts that substantially interfere with the methods of the present invention.
Locus—A location on a chromosome or DNA molecule corresponding to a gene or a physical or phenotypic feature, where physical features include polymorphic sites.
Nucleotide pair—The nucleotides found at a polymorphic site on the two copies of a chromosome from an individual.
Phased—As applied to a sequence of nucleotide pairs for two or more polymorphic sites in a locus, phased means the combination of nucleotides present at those polymorphic sites on a single copy of the locus is known.
Polymorphic site (PS)—A position on a chromosome or DNA molecule at which at least two alternative sequences are found in a population.
Polymorphism—The sequence variation observed in an individual at a polymorphic site. Polymorphisms include nucleotide substitutions, insertions, deletions and microsatellites and may, but need not, result in detectable differences in gene expression or protein function.
Polynucleotide—A nucleic acid molecule comprised of single-stranded RNA or DNA or comprised of complementary, double-stranded DNA.
Population Group—A group of individuals sharing a common ethnogeographic origin.
Reference Population—A group of subjects or individuals who are predicted to be representative of the genetic variation found in the general population. Typically, the reference population represents the genetic variation in the population at a certainty level of at least 85%, preferably at least 90%, more preferably at least 95% and even more preferably at least 99%.
Single Nucleotide Polymorphism (SNP)—Typically, the specific pair of nucleotides observed at a single polymorphic site. In rare cases, three or four nucleotides may be found.
Subject—A human individual whose genotypes or haplotypes or age of onset to treatment or disease state are to be determined.
Treatment—A stimulus administered internally or externally to a subject.
Unphased—As applied to a sequence of nucleotide pairs for two or more polymorphic sites in a locus, unphased means the combination of nucleotides present at those polymorphic sites on a single copy of the locus is not known.

### DESCRIPTION OF THE PREFERRED EMBODIMENTS

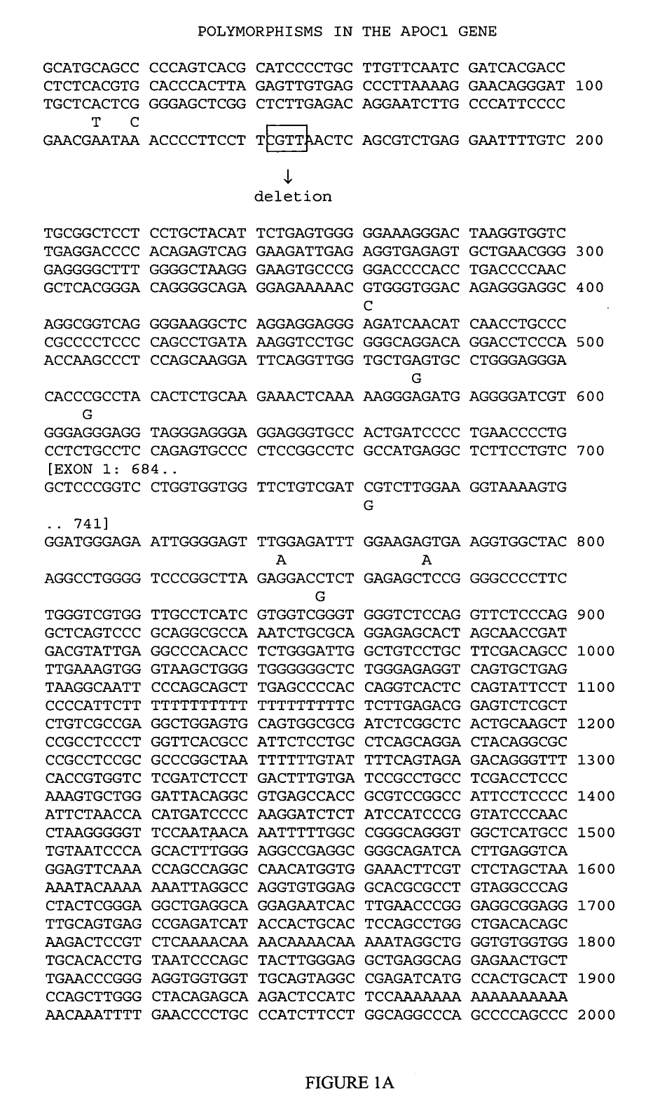

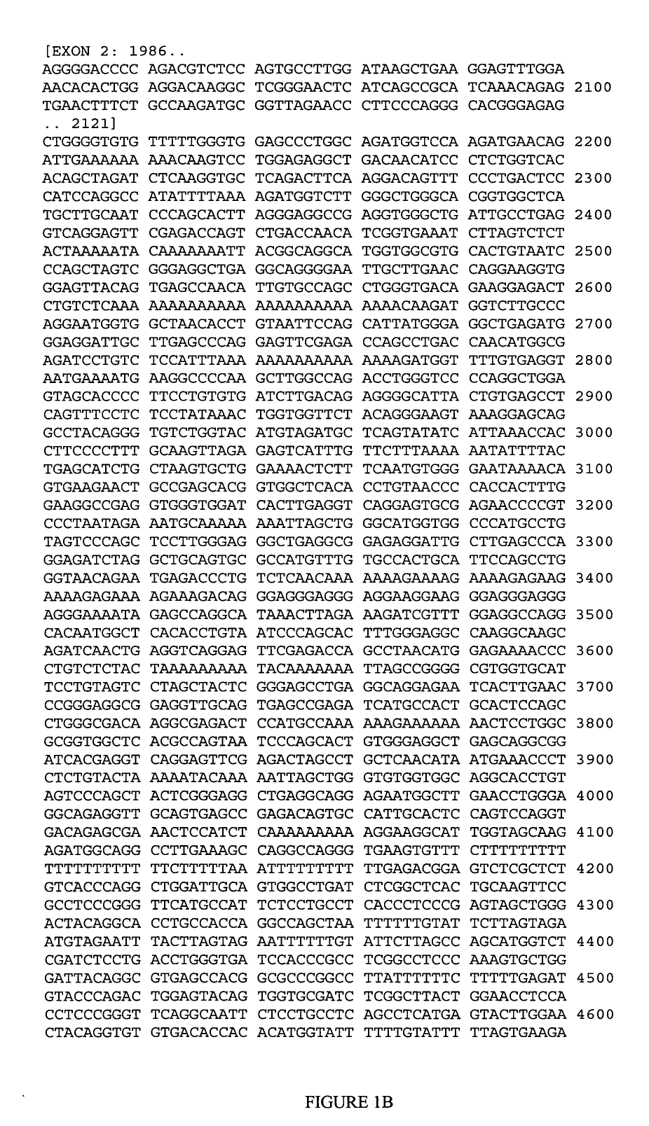

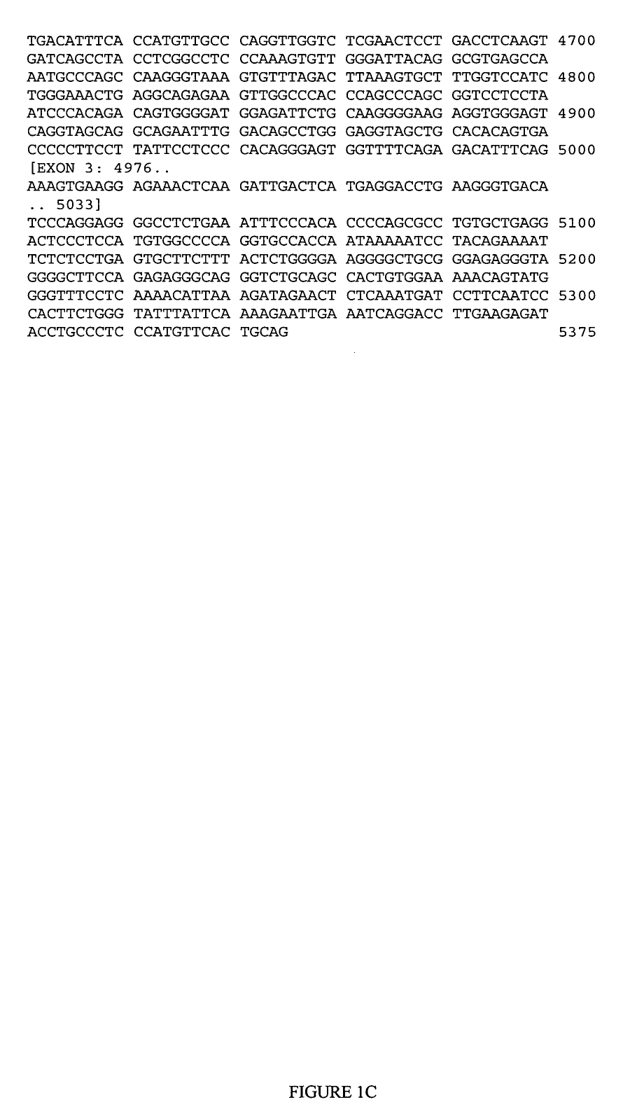

Each age of onset marker of the invention is a combination of a particular haplotype and the copy number for that haplotype. Preferably, the haplotype is one of the haplotypes shown in Table 1. The PS or PSs in these haplotypes are referred to herein as PS1, PS2, PS3, PS4, PS5, PS6, PS7, PS8, PS9, and PS10 and are located in the APOC1 gene at positions corresponding to those identified in FIG. 1/SEQ ID NO: 1 (see Table 2 for summary of PS1, PS2, PS3, PS4, PS5, PS6, PS7, PS8, PS9, and PS10 and locations). In describing the PSs in the age of onset markers of the invention, reference is made to the sense strand of a gene for convenience. However, as recognized by the skilled artisan, nucleic acid molecules containing a particular gene may be complementary double stranded molecules and thus reference to a particular site or haplotype on the sense strand refers as well to the corresponding site or haplotype on the complementary antisense strand. Further, reference may be made to detecting a genetic marker or haplotype for one strand and it will be understood by the skilled artisan that this includes detection of the complementary haplotype on the other strand.

As described in more detail in the examples below, the age of onset markers of the invention are based on the discovery by the inventors of associations between certain haplotypes in the APOC1 gene and the age of onset of AD in a cohort of individuals diagnosed with AD.

In particular, the inventors herein discovered that a haplotype comprising a deletion of CGTT at PS3 (haplotype (23) in Table 1) affected the age of onset of AD of the patients participating in the study. The group of patients having one copy or two copies of this haplotype experienced a later age of onset of AD than the patient group having zero copies of the haplotype.

In addition, the skilled artisan would expect that there might be additional PSs in the APOC1 gene or elsewhere on chromosome 19, wherein an allele at that PS is in high linkage disequilibrium (LD) with an allele at one or more of the PSs in the haplotypes comprising an age of onset marker I or an age of onset marker II. Two particular alleles at different PSs are said to be in LD if the presence of the allele at one of the sites tends to predict the presence of the allele at the other site on the same chromosome (Stevens, Mol. Diag. 4:309-17 (1999)). One of the most frequently used measures of linkage disequilibrium is Δ², which is calculated using the formula described by Devlin et al. (Genomics 29(2):3112-22 (1995)). Δ² is the measure of how well an allele X at a first PS predicts the occurrence of an allele Y at a second PS on the same chromosome. The measure only reaches 1.0 when the prediction is perfect (e.g., X if and only if Y).

Thus, the skilled artisan would expect that all of the embodiments of the invention described herein may frequently be practiced by substituting any (or all) of the specifically identified APOC1 PSs in an age of onset marker with another PS, wherein an allele at the substituted PS is in LD with an allele at the “substituting” PS. This “substituting” PS may be one that is currently known or subsequently discovered and may be present in the APOC1 gene, in a genomic region of about 100 kilobases spanning the APOC1 gene, or elsewhere on chromosome 19.

Further, the inventors contemplate that there will be other haplotypes in the APOC1 gene or elsewhere on chromosome 19 that are in LD with one or more of the haplotypes in Table 1 that would therefore also be predictive of age of onset of AD. Preferably, the linked haplotype is present in the APOC1 gene or in a genomic region of about 100 kilobases spanning the APOC1 gene. The linkage disequilibrium between the haplotypes in Table 1 and such linked haplotypes can also be measured using Δ².

In preferred embodiments, the linkage disequilibrium between an allele at a polymorphic site in any of the haplotypes in Table 1 and an allele at a “substituting” polymorphic site, or between any of the haplotypes in Table 1 and a linked haplotype, has a Δ² value, as measured in a suitable reference population, of at least 0.75, more preferably at least 0.80, even more preferably at least 0.85 or at least 0.90, yet more preferably at least 0.95, and most preferably 1.0. A suitable reference population for this Δ² measurement is selected from a population with the distribution of its members reflecting the general population, a population with AD or AD risk factors, and the like.

LD patterns in genomic regions are readily determined empirically in appropriately chosen samples using various techniques known in the art for determining whether any two alleles (either those occurring at two different PSs or two haplotypes for two different multi-site loci) are in linkage disequilibrium (GENETIC DATA ANALYSIS II, Weir, Sinauer Associates, Inc. Publishers, Sunderland, Mass., 1996). The skilled artisan may readily select which method of determining LD will be best suited for a particular sample size and genomic region.

As described above and in the examples below, the age of onset markers of the invention are associated with differences in the age of onset of AD. Thus, the invention provides a method and kit for determining whether an individual has an age of onset marker I or an age of onset marker II. An age of onset marker I is (a) one copy or two copies of any of (i) haplotypes (1)-(64) in Table 1, (ii) a linked haplotype for any of haplotypes (1)-(64) in Table 1, and (iii) a substitute haplotype for any of haplotypes (1)-(64) in Table 1, or (b) zero copies or one copy of any of (i) haplotypes (65)-(68), (ii) a linked haplotype for any of haplotypes (65)-(68) in Table 1, and (iii) a substitute haplotype for any of haplotypes (65)-(68) in Table 1. An age of onset marker II is (a) zero copies of any of (i) haplotypes (1)-(64) in Table 1, (ii) a linked haplotype for any of haplotypes (1)-(64) in Table 1, and (iii) a substitute haplotype for any of haplotypes (1)-(64) in Table 1, or (b) two copies of any of (i) haplotypes (65)-(68), (ii) a linked haplotype for any of haplotypes (65)-(68) in Table 1, and (iii) a substitute haplotype for any of haplotypes (65)-(68) in Table 1.

In one embodiment, the invention provides a method for determining whether an individual has an age of onset marker I or an age of onset marker II. The method comprises determining whether the individual has (a) one copy or two copies, or zero copies of any of (i) haplotypes (1)-(64) in Table 1, (ii) a linked haplotype for any of haplotypes (1)-(64) in Table 1, and (iii) a substitute haplotype for any of haplotypes (1)-(64) in Table 1, or (b) zero copies or one copy, or two copies of any of (i) haplotypes (65)-(68), (ii) a linked haplotype for any of haplotypes (65)-(68) in Table 1, and (iii) a substitute haplotype for any of haplotypes (65)-(68) in Table 1. Preferably, the method comprises determining whether the individual has one copy or two copies, or zero copies of any of (a) haplotype (23) in Table 1, (b) a linked haplotype for haplotype (23) in Table 1, and (c) a substitute haplotype for haplotype (23) in Table 1.

In some embodiments, the individual is Caucasian and is at risk for developing a cognitive disorder, such as mild to moderate dementia of the Alzheimer's type, dementia associated with Parkinson's Disease, MCI, a vascular dementia, and Lewy body dementia.

In another embodiment, the invention provides a method for assigning an individual to a first or second age of onset marker group. The method comprises determining whether the individual has (a) one copy or two copies, or zero copies of any of (i) haplotypes (1)-(64) in Table 1, (ii) a linked haplotype for any of haplotypes (1)-(64) in Table 1, and (iii) a substitute haplotype for any of haplotypes (1)-(64) in Table 1, or (b) zero copies or one copy, or two copies of any of (i) haplotypes (65)-(68), (ii) a linked haplotype for any of haplotypes (65)-(68) in Table 1, and (iii) a substitute haplotype for any of haplotypes (65)-(68) in Table 1, and assigning the individual to the first age of onset marker group if the individual has (a) one copy or two copies of any of (i) haplotypes (1)-(64) in Table 1, (ii) a linked haplotype for any of haplotypes (1)-(64) in Table 1, and (iii) a substitute haplotype for any of haplotypes (1)-(64) in Table 1, or (b) zero copies or one copy of any of (i) haplotypes (65)-(68), (ii) a linked haplotype for any of haplotypes (65)-(68) in Table 1, and (iii) a substitute haplotype for any of haplotypes (65)-(68) in Table 1, and assigning the individual to the second age of onset marker group if the individual has (a) zero copies of any of (i) haplotypes (1)-(64) in Table 1, (ii) a linked haplotype for any of haplotypes (1)-(64) in Table 1, and (iii) a substitute haplotype for any of haplotypes (1)-(64) in Table 1, or (b) two copies of any of (i) haplotypes (65)-(68), (ii) a linked haplotype for any of haplotypes (65)-(68) in Table 1, and (iii) a substitute haplotype for any of haplotypes (65)-(68) in Table 1.

In some embodiments, the individual is Caucasian and is at risk for developing a cognitive disorder, such as mild to moderate dementia of the Alzheimer's type, dementia associated with Parkinson's Disease, MCI, a vascular dementia, and Lewy body dementia.

The presence in an individual of an age of onset marker I or an age of onset marker II may be determined by a variety of indirect or direct methods well known in the art for determining haplotypes or haplotype pairs for a set of one or more PSs in one or both copies of the individual's genome, including those discussed below. The genotype for a PS in an individual may be determined by methods known in the art or as described below.

One indirect method for determining whether zero copies, one copy, or two copies of a haplotype is present in an individual is by prediction based on the individual's genotype determined at one or more of the PSs comprising the haplotype and using the determined genotype at each site to determine the haplotypes present in the individual. The presence of zero copies, one copy, or two copies of a haplotype of interest can be determined by visual inspection of the alleles at the PS that comprise the haplotype. The haplotype pair is assigned by comparing the individual's genotype with the genotypes at the same set of PS corresponding to the haplotype pairs known to exist in the general population or in a specific population group or to the haplotype pairs that are theoretically possible based on the alternative alleles possible at each PS, and determining which haplotype pair is most likely to exist in the individual.

In a related indirect haplotyping method, the presence in an individual of zero copies, one copy, or two copies of a haplotype is predicted from the individual's genotype for a set of PSs comprising the selected haplotype using information on haplotype pairs known to exist in a reference population. In one embodiment, this haplotype pair prediction method comprises identifying a genotype for the individual at the set of PSs comprising the selected haplotype, accessing data containing haplotype pairs identified in a reference population for a set of PSs comprising the PSs of the selected haplotype, and assigning to the individual a haplotype pair that is consistent with the individual's genotype. Whether the individual has an age of onset marker I or an age of onset marker II can be subsequently determined based on the assigned haplotype pair. The haplotype pair can be assigned by comparing the individual's genotype with the genotypes corresponding to the haplotype pairs known to exist in the general population or in a specific population group, and determining which haplotype pair is consistent with the genotype of the individual. In some embodiments, the comparing step may be performed by visual inspection. When the genotype of the individual is consistent with more than one haplotype pair, frequency data may be used to determine which of these haplotype pairs is most likely to be present in the individual. If a particular haplotype pair consistent with the genotype of the individual is more frequent in the reference population than other pairs consistent with the genotype, then that haplotype pair with the highest frequency is the most likely to be present in the individual. The haplotype pair frequency data used in this determination is preferably for a reference population comprising the same ethnogeographic group as the individual. This determination may also be performed in some embodiments by visual inspection. In other embodiments, the comparison may be made by a computer-implemented algorithm with the genotype of the individual and the reference haplotype data stored in computer-readable formats. For example, as described in WO 01/80156, one computer-implemented algorithm to perform this comparison entails enumerating all possible haplotype pairs which are consistent with the genotype, accessing data containing haplotype pairs frequency data determined in a reference population to determine a probability that the individual has a possible haplotype pair, and analyzing the determined probabilities to assign a haplotype pair to the individual.

Typically, the reference population is composed of randomly selected individuals representing the major ethnogeographic groups of the world. A preferred reference population for use in the methods of the present invention consists of Caucasian individuals, the number of which is chosen based on how rare a haplotype is that one wants to be guaranteed to see. For example, if one wants to have a q % chance of not missing a haplotype that exists in the population at a p % frequency of occurring in the reference population, the number of individuals (n) who must be sampled is given by 2n=log(1−q)/log(1−p) where p and q are expressed as fractions. A preferred reference population allows the detection of any haplotype whose frequency is at least 10% with about 99% certainty. A particularly preferred reference population includes a 3-generation Caucasian family to serve as a control for checking quality of haplotyping procedures.

If the reference population comprises more than one ethnogeographic group, the frequency data for each group is examined to determine whether it is consistent with Hardy-Weinberg equilibrium. Hardy-Weinberg equilibrium (PRINCIPLES OF POPULATION GENOMICS, 3^{rd} ed., Hartl, Sinauer Associates, Sunderland, Mass., 1997) postulates that the frequency of finding the haplotype pair H₁/H₂is equal to p_{H-W}(H₁/H₂ )=2p(U₁)p(H₂) if H₁≠H₂ and P_{H-W}(H₁/H₂)=p(H₁)p(H₂) if H₁=H₂. A statistically significant difference between the observed and expected haplotype frequencies could be due to one or more factors including significant inbreeding in the population group, strong selective pressure on the gene, sampling bias, and/or errors in the genotyping process. If large deviations from Hardy-Weinberg equilibrium are observed in an ethnogeographic group, the number of individuals in that group can be increased to see if the deviation is due to a sampling bias. If a larger sample size does not reduce the difference between observed and expected haplotype pair frequencies, then one may wish to consider haplotyping the individual using a direct haplotyping method such as, for example, CLASPER System™ technology ((U.S. Pat. No. 5,866,404), single molecule dilution, or allele-specific long-range PCR (Michalotos-Beloin et al., Nucleic Acids Res. 24:4841-3 (1996)).

In one embodiment of this method for predicting a haplotype pair for an individual, the assigning step involves performing the following analysis. First, each of the possible haplotype pairs is compared to the haplotype pairs in the reference population. Generally, only one of the haplotype pairs in the reference population matches a possible haplotype pair and that pair is assigned to the individual. Occasionally, only one haplotype represented in the reference haplotype pairs is consistent with a possible haplotype pair for an individual, and in such cases the individual is assigned a haplotype pair containing this known haplotype and a new haplotype derived by subtracting the known haplotype from the possible haplotype pair. Alternatively, the haplotype pair in an individual may be predicted from the individual's genotype for that gene using reported methods (e.g., Clark et al., Mol. Biol. Evol. 7:1121-22 (1990) or WO 01/80156) or through a commercial haplotyping service such as offered by Genaissance Pharmaceuticals, Inc. (New Haven, Conn.). In rare cases, either no haplotypes in the reference population are consistent with the possible haplotype pairs, or alternatively, multiple reference haplotype pairs are consistent with the possible haplotype pairs. In such cases, the individual is preferably haplotyped using a direct molecular haplotyping method such as, for example, CLASPER System™ technology (U.S. Pat. No. 5,866,404), SMD, or allele-specific long-range PCR (Michalotos-Beloin et al., supra).

Determination of the number of haplotypes present in the individual from the genotypes is illustrated here for haplotype (23) in Table 1. Table 3 below shows the 3 (3ⁿ, where each of n bi-allelic polymorphic sites may have one of 3 different genotypes present) genotypes that may be detected at PS3, using both chromosomal copies from an individual. Each of the three possible genotypes allow unambiguous determination of the number of copies of the haplotype (23) in Table 1 present in the individual and therefore would allow unambiguous determination of whether the individual has an age of onset marker I or an age of onset marker II. For instances where there is ambiguity in the haplotype pair underlying the determined genotype (i.e., when two or more PSs are included in the haplotype), frequency information may be used to determine the most probable haplotype pair and therefore the most likely number of copies of the haplotype in the individual. If a particular haplotype pair consistent with the genotype of the individual is more frequent in the reference population than other pairs consistent with the genotype, then that haplotype pair with the highest frequency is the most likely to be present in the individual. The copy number of the haplotype of interest in this haplotype pair can then be determined by visual inspection of the alleles at the PS that comprise the age of onset marker for each haplotype in the pair.

Alternatively, for the ambiguous genotypes, genotyping of one or more additional sites in APOC1 may be performed to eliminate the ambiguity in deconvoluting the haplotype pairs underlying the genotype at the particular PSs. The skilled artisan would recognize that alleles at these one or more additional sites would need to have sufficient linkage with the alleles in at least one of the possible haplotypes in the pair to permit unambiguous assignment of the haplotype pair. Although this illustration has been directed to the particular instance of determining the number of copies of haplotype (23) in Table 1 present in an individual, the process would be analogous for the other haplotypes shown in Table 1, or for the linked haplotypes or substitute haplotypes for any of the haplotypes in Table 1.

TABLE 3
Possible Copy Numbers of Haplotype (23) in Table 1
Based on Genotypes at PS3

| | Copy Number |
|---|---|
| | of Haploytpe |
| PS3 | (23) in Table 1 |
| —/— | 0 |
| —/ | 1 |
| deletion | |
| of CGTT | |
| deletion | 2 |
| of CGTT/ | |
| deletion | |
| of CGTT | |

The individual's genotype for the desired set of PS may be determined using a variety of methods well-known in the art. Such methods typically include isolating from the individual a genomic DNA sample comprising both copies of the gene or locus of interest, amplifying from the sample one or more target regions containing the polymorphic sites to be genotyped, and detecting the nucleotide pair present at each PS of interest in the amplified target region(s). It is not necessary to use the same procedure to determine the genotype for each PS of interest.

In addition, the identity of the allele(s) present at any of the novel PSs described herein may be indirectly determined by haplotyping or genotyping another PS having an allele that is in linkage disequilibrium with an allele of the PS that is of interest. PSs having an allele in linkage disequilibrium with an allele of the presently disclosed PSs may be located in regions of the gene or in other genomic regions not examined herein. Detection of the allele(s) present at a PS, wherein the allele is in linkage disequilibrium with an allele of the novel PSs described herein may be performed by, but is not limited to, any of the above-mentioned methods for detecting the identity of the allele at a PS.

Alternatively, the presence in an individual of a haplotype or haplotype pair for a set of PSs comprising an age of onset marker may be determined by directly haplotyping at least one of the copies of the individual's genomic region of interest, or suitable fragment thereof, using methods known in the art. Such direct haplotyping methods typically involve treating a genomic nucleic acid sample isolated from the individual in a manner that produces a hemizygous DNA sample that only has one of the two “copies” of the individual's genomic region which, as readily understood by the skilled artisan, may be the same allele or different alleles, amplifying from the sample one or more target regions containing the PSs to be genotyped, and detecting the nucleotide present at each PS of interest in the amplified target region(s). The nucleic acid sample may be obtained using a variety of methods known in the art for preparing hemizygous DNA samples, which include: targeted in vivo cloning (TIVC) in yeast as described in WO 98/01573, U.S. Pat. No. 5,866,404, and U.S. Pat. No. 5,972,614; generating hemizygous DNA targets using an allele specific oligonucleotide in combination with primer extension and exonuclease degradation as described in U.S. Pat. No. 5,972,614; single molecule dilution (SMD) as described in Ruaño et al., Proc. Natl. Acad. Sci. 87:6296-300 (1990); and allele specific PCR (Ruaño et al., Nucl. Acids Res. 17:8392 (1989); Ruaño et al., Nucl. Acids Res. 19:6877-82 (1991); Michalatos-Beloin et al., supra).

As will be readily appreciated by those skilled in the art, any individual clone will typically only provide haplotype information on one of the two genomic copies present in an individual. If haplotype information is desired for the individual's other copy, additional clones will usually need to be examined. Typically, at least five clones should be examined to have more than a 90% probability of haplotyping both copies of the genomic locus in an individual. In some cases, however, once the haplotype for one genomic allele is directly determined, the haplotype for the other allele may be inferred if the individual has a known genotype for the PSs of interest or if the haplotype frequency or haplotype pair frequency for the individual's population group is known.

While direct haplotyping of both copies of the gene is preferably performed with each copy of the gene being placed in separate containers, it is also envisioned that direct haplotyping could be performed in the same container if the two copies are labeled with different tags, or are otherwise separately distinguishable or identifiable. For example, if first and second copies of the gene are labeled with different first and second fluorescent dyes, respectively, and an allele-specific oligonucleotide labeled with yet a third different fluorescent dye is used to assay the PS(s), then detecting a combination of the first and third dyes would identify the polymorphism in the first gene copy while detecting a combination of the second and third dyes would identify the polymorphism in the second gene copy.

The nucleic acid sample used in the above indirect and direct haplotyping methods is typically isolated from a biological sample taken from the individual, such as a blood sample or tissue sample. Suitable tissue samples include whole blood, saliva, tears, urine, skin and hair.

The target region(s) containing the PS of interest may be amplified using any oligonucleotide-directed amplification method, including but not limited to polymerase chain reaction (PCR) (U.S. Pat. No. 4,965,188), ligase chain reaction (LCR) (Barany et al., Proc. Natl. Acad. Sci. USA 88:189-93 (1991); WO 90/01069), and oligonucleotide ligation assay (OLA) (Landegren et al., Science 241:1077-80 (1988)). Other known nucleic acid amplification procedures may be used to amplify the target region(s) including transcription-based amplification systems (U.S. Pat. No. 5,130,238; European Patent No. EP 329,822; U.S. Pat. No. 5,169,766; WO 89/06700) and isothermal methods (Walker et al., Proc. Natl. Acad. Sci. USA 89:392-6 (1992)).

In both the direct and indirect haplotyping methods, the identity of a nucleotide (or nucleotide pair) at a PS(s) in the amplified target region may be determined by sequencing the amplified region(s) using conventional methods. If both copies of the gene are represented in the amplified target, it will be readily appreciated by the skilled artisan that only one nucleotide will be detected at a PS in individuals who are homozygous at that site, while two different nucleotides will be detected if the individual is heterozygous for that site. The polymorphism may be identified directly, known as positive-type identification, or by inference, referred to as negative-type identification. For example, where a polymorphism is known to be guanine and cytosine in a reference population, a site may be positively determined to be either guanine or cytosine for an individual homozygous at that site, or both guanine and cytosine, if the individual is heterozygous at that site. Alternatively, the site may be negatively determined to be not guanine (and thus cytosine/cytosine) or not cytosine (and thus guanine/guanine).

A PS in the target region may also be assayed before or after amplification using one of several hybridization-based methods known in the art. Typically, allele-specific oligonucleotides are utilized in performing such methods. The allele-specific oligonucleotides may be used as differently labeled probe pairs, with one member of the pair showing a perfect match to one variant of a target sequence and the other member showing a perfect match to a different variant. In some embodiments, more than one PS may be detected at once using a set of allele-specific oligonucleotides or oligonucleotide pairs. Preferably, the members of the set have melting temperatures within 5° C., and more preferably within 2° C., of each other when hybridizing to each of the polymorphic sites being detected.

Hybridization of an allele-specific oligonucleotide to a target polynucleotide may be performed with both entities in solution, or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking baking, etc. Allele-specific oligonucleotides may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibers, chips, dishes, and beads. The solid support may be treated, coated or derivatized to facilitate the immobilization of the allele-specific oligonucleotide or target nucleic acid.

Detecting the nucleotide or nucleotide pair at a PS of interest may also be determined using a mismatch detection technique, including but not limited to the RNase protection method using riboprobes (Winter et al., Proc. Natl. Acad. Sci. USA 82:7575 (1985); Meyers et al., Science 230:1242 (1985)) and proteins which recognize nucleotide mismatches, such as the E. coli mutS protein (Modrich, Ann. Rev. Genet. 25:229-53 (1991)). Alternatively, variant alleles can be identified by single strand conformation polymorphism (SSCP) analysis (Orita et al., Genomics 5:874-9 (1989); Humphries et al., in MOLECULAR DIAGNOSIS OF GENETIC DISEASES, Elles, ed., pp. 321-340, 1996) or denaturing gradient gel electrophoresis (DGGE) (Wartell et al., Nucl. Acids Res. 18:2699-706 (1990); Sheffield et al., Proc. Natl. Acad. Sci. USA 86:232-6 (1989)).

A polymerase-mediated primer extension method may also be used to identify the polymorphism(s). Several such methods have been described in the patent and scientific literature and include the “Genetic Bit Analysis” method (WO 92/15712) and the ligase/polymerase mediated genetic bit analysis (U.S. Pat. No. 5,679,524. Related methods are disclosed in WO 91/02087, WO 90/09455, WO 95/17676, and U.S. Pat. Nos. 5,302,509 and 5,945,283. Extended primers containing the complement of the polymorphism may be detected by mass spectrometry as described in U.S. Pat. No. 5,605,798. Another primer extension method is allele-specific PCR (Ruaño et al., 1989, supra; Ruaño et al., 1991, supra; WO 93/22456; Turki et al., J. Clin. Invest. 95:1635-41 (1995)). In addition, multiple PSs may be investigated by simultaneously amplifying multiple regions of the nucleic acid using sets of allele-specific primers as described in WO 89/10414.

The genotype or haplotype for the APOC1 gene of an individual may also be determined by hybridization of a nucleic acid sample containing one or both copies of the gene, mRNA, cDNA or fragment(s) thereof, to nucleic acid arrays and subarrays such as described in WO 95/112995. The arrays would contain a battery of allele-specific oligonucleotides representing each of the PSs to be included in the genotype or haplotype.

The invention also provides a kit for determining whether an individual has an age of onset marker I or an age of onset marker II. The kit comprises a set of one or more oligonucleotides designed for identifying at least one of the alleles at each PS in a set of one or more PSs, wherein the set of one or more PSs comprises (a) PS3 and PS7; (b) PS3, PS5, PS7, and PS9; (c) PS1, PS3, PS7, and PS9; (d) PS3, PS6, PS7, and PS9; (e) PS2, PS3, PS7, and PS9; (f) PS3, PS7, PS9, and PS10; (g) PS2, PS3, PS5, and PS7; (h) PS3, PS5, and PS7; (i) PS1, PS3, and PS7; )PS2, PS3, and PS7; (k) PS3, PS6, and PS7; (1) PS3, PS7, and PS10; (m) PS1, PS3, PS5, and PS7; (n) PS3, PS5, PS6, and PS7; (o) PS1, PS3, PS6, and PS7; (p) PS1, PS2, PS3, and PS7; (q)PS2, PS3, PS6, and PS7; (r) PS3, PS5, PS7, and PS10; (s) PS1, PS3, PS7, and PS10; (t) PS3, PS7, and PS9; (u) PS3, PS6, PS7, and PS10; (v) PS2, PS3, PS7, and PS10; (w) PS3; (x) PS3, PS5, PS6, and PS9; (y) PS3, PS5, and PS10; (z) PS2, PS3, PS9, and PS10; (aa) PS1, PS3, PS6, and PS10; (bb) PS1, PS3, and PS5; (cc) PS1, PS2, PS3, and PS9; (dd) PS3, PS6, and PS9; (ee) PS3 and PS10; (ff) PS2, PS3, PS5, and PS9; (gg) PS1 and PS3; (hh) PS3, PS5, PS6, and PS10; (ii) PS1, PS3, PS5, and PS6; (jj) PS1, PS2, PS3, and PS10; (kk) PS3 and PS5; (ll) PS3, PS6, and PS10; (mm) PS2, PS3, and PS9; (nn) PS1, PS3, and PS6; (oo) PS2, PS3, PS5, and PS10; (pp) PS1, PS2, PS3, and PS5; (qq) PS3, PS5, and PS6; (rr) PS1, PS3, PS9, and PS10; (ss) PS2, PS3, PS6, and PS9; (tt) PS2, PS3, and PS10; (uu) PS1, PS2, and PS3; (vv) PS3 and PS6; (ww) PS3, PS5, PS9, and PS10; (xx) PS1, PS3, PS5, and PS9; (yy) PS2, PS3, and PS5; (zz) PS2, PS3, PS6, and PS10; (aaa) PS3, PS9, and PS10; (bbb) PS1, PS2, PS3, and PS6; (ccc) PS1, PS3, and PS9; (ddd) PS2 and PS3; (eee) PS2, PS3, PS5, and PS6; (fff) PS1, PS3, PS5, and PS10; (ggg) PS3, PS6, PS9, and PS10; (hhh) PS3, PS5, and PS9; (iii) PS1, PS3, PS6, and PS9; (jjj) PS1, PS3, and PS10; (kkk) PS2, PS3, and PS6; (lll) PS3 and PS9; (mmm) PS3; (nnn) PS3 and PS7; (ooo) PS1 and PS3; (ppp) PS1, PS3, and PS7; (qqq) a set of one or more PSs in a linked haplotype for any of haplotypes (1)-(68) in Table 1, or (rrr) a set of one or more PSs in a substitute haplotype for any of haplotypes (1)-(68) in Table 1. Preferably, the kit comprises a set of one or more oligonucleotides designed for identifying at least one of the alleles at each PS in a set of one or more PSs, wherein the set of one or more PSs is any of (a) PS3 and PS7; (b) PS3, PS5, PS7, and PS9; (c) PS1, PS3, PS7, and PS9; (d) PS3, PS6, PS7, and PS9; (e) PS2, PS3, PS7, and PS9; (f) PS3, PS7, PS9, and PS10; (g) PS2, PS3, PS5, and PS7; (h) PS3, PS5, and PS7; (i) PS1, PS3, and PS7; (j) PS2, PS3, and PS7; (k) PS3, PS6, and PS7; (l) PS3, PS7, and PS10; (m) PS1, PS3, PS5, and PS7; (n) PS3, PS5, PS6, and PS7; (o) PS1, PS3, PS6, and PS7; (p) PS1, PS2, PS3, and PS7; (q) PS2, PS3, PS6, and PS7; (r) PS3, PS5, PS7, and PS10; (s) PS1, PS3, PS7, and PS10; (t) PS3, PS7, and PS9; (u) PS3, PS6, PS7, and PS10; (v) PS2, PS3, PS7, and PS10; (w) PS3; (x) PS3, PS5, PS6, and PS9; (y) PS3, PS5, and PS10; (z) PS2, PS3, PS9, and PS10; (aa) PS1, PS3, PS6, and PS10; (bb) PS1, PS3, and PS5; (cc) PS1, PS2, PS3, and PS9; (dd) PS3, PS6, and PS9; (ee) PS3 and PS10; (ff) PS2, PS3, PS5, and PS9; (gg) PS1 and PS3; (hh) PS3, PS5, PS6, and PS10; (ii) PS1, PS3, PS5, and PS6; (jj) PS1, PS2, PS3, and PS10; (kk) PS3 and PS5; (ll) PS3, PS6, and PS10; (mm) PS2, PS3, and PS9; (nn) PS1, PS3, and PS6; (oo) PS2, PS3, PS5, and PS10; (pp) PS1, PS2, PS3, and PS5; (qq) PS3, PS5, and PS6; (rr) PS1, PS3, PS9, and PS10; (ss) PS2, PS3, PS6, and PS9; (tt) PS2, PS3, and PS10; (uu) PS1, PS2, and PS3; (vv) PS3 and PS6; (ww) PS3, PS5, PS9, and PS10; (xx) PS1, PS3, PS5, and PS9; (yy) PS2, PS3, and PS5; (zz) PS2, PS3, PS6, and PS10; (aaa) PS3, PS9, and PS 10; (bbb) PS1, PS2, PS3, and PS6; (ccc) PS1, PS3, and PS9; (ddd) PS2 and PS3; (eee) PS2, PS3, PS5, and PS6; (fff) PS1, PS3, PS5, and PS10; (ggg) PS3, PS6, PS9, and PS10; (hhh) PS3, PS5, and PS9; (iii) PS1, PS3, PS6, and PS9; (jj) PS1, PS3, and PS10; (kkk) PS2, PS3, and PS6; (lll) PS3 and PS9; (mmm) PS3; (nnn) PS3 and PS7; (ooo) PS1 and PS3; (ppp) PS1, PS3, and PS7; (qqq) a set of one or more PSs in a linked haplotype for any of haplotypes (1)-(68) in Table 1, and (rrr) a set of one or more PSs in a substitute haplotype for any of haplotypes (I)-(68) in Table 1.

In a preferred embodiment of the kit of the invention, the set of one or more oligonucleotides is designed for identifying both alleles at each PS in the set of one or more PSs. In another preferred embodiment, the individual is Caucasian. In another preferred embodiment, the kit further comprises a manual with instructions for (a) performing one or more reactions on a human nucleic acid sample to identify the allele or alleles present in the individual at each PS in the set of one or more PSs, and (b) determining if the individual has an age of onset marker I or an age of onset marker II based on the identified allele or alleles. In another preferred embodiment, the linkage disequilibrium between the linked haplotype and at least one of haplotypes (1)-(68) in Table 1 has a delta squared value selected from the group consisting of at least 0.75, at least 0.80, at least 0.85, at least 0.90, at least 0.95, and 1.0. In yet another preferred embodiment, the linkage disequilibrium between the allele at a substituting PS in the substitute haplotype and the allele at a substituted PS in any of haplotypes (1)-(68) in Table 1 has a delta squared value selected from the group consisting of at least 0.75, at least 0.80, at least 0.85, at least 0.90, at least 0.95, and 1.0.

As used herein, an “oligonucleotide” is a probe or primer capable of hybridizing to a target region that contains, or that is located close to, a PS of interest. Preferably, the oligonucleotide has less than about 100 nucleotides. More preferably, the oligonucleotide is 10 to 35 nucleotides long. Even more preferably, the oligonucleotide is between 15 and 30, and most preferably, between 20 and 25 nucleotides in length. The exact length of the oligonucleotide will depend on the nature of the genomic region containing the PS as well as the genotyping assay to be performed and is readily determined by the skilled artisan.

The oligonucleotides used to practice the invention may be comprised of any phosphorylation state of ribonucleotides, deoxyribonucleotides, and acyclic nucleotide derivatives, and other functionally equivalent derivatives.

Alternatively, oligonucleotides may have a phosphate-free backbone, which may be comprised of linkages such as carboxymethyl, acetamidate, carbamate, polyamide (peptide nucleic acid (PNA)) and the like (Varma, in MOLECULAR BIOLOGY AND BIOTECHNOLOGY, A COMPREHENSIVE DESK REFERENCE, Meyers, ed., pp. 617-20, VCH Publishers, Inc., 1995). Oligonucleotides of the invention may be prepared by chemical synthesis using any suitable methodology known in the art, or may be derived from a biological sample, for example, by restriction digestion. The oligonucleotides may be labeled, according to any technique known in the art, including use of radiolabels, fluorescent labels, enzymatic labels, proteins, haptens, antibodies, sequence tags and the like.

Oligonucleotides of the invention must be capable of specifically hybridizing to a target region of a polynucleotide containing a desired locus.

As used herein, specific hybridization means the oligonucleotide forms an anti-parallel double-stranded structure with the target region under certain hybridizing conditions, while failing to form such a structure when incubated with another region in the polynucleotide or with a polynucleotide lacking the desired locus under the same hybridizing conditions. Preferably, the oligonucleotide specifically hybridizes to the target region under conventional high stringency conditions.

A nucleic acid molecule such as an oligonucleotide or polynucleotide is said to be a “perfect” or “complete” complement of another nucleic acid molecule if every nucleotide of one of the molecules is complementary to the nucleotide at the corresponding position of the other molecule. A nucleic acid molecule is “substantially complementary” to another molecule if it hybridizes to that molecule with sufficient stability to remain in a duplex form under conventional low-stringency conditions. Conventional hybridization conditions are described, for example, in MOLECULAR CLONING, A LABORATORY MANUAL, 2^{nd} ed., Sambrook et al., Cold Spring Harbor Press, Cold Spring Harbor, N.Y., 1989, and in NUCLEIC ACID HYBRIDIZATION, A PRACTICAL APPROACH, Haymes et al., IRL Press, Washington, D.C., 1985. While perfectly complementary oligonucleotides are preferred for detecting polymorphisms, departures from complete complementarity are contemplated where such departures do not prevent the molecule from specifically hybridizing to the target region. For example, an oligonucleotide primer may have a non-complementary fragment at its 5′ end, with the remainder of the primer being complementary to the target region. Alternatively, non-complementary nucleotides may be interspersed into the probe or primer as long as the resulting probe or primer is still capable of specifically hybridizing to the target region.

Preferred oligonucleotides of the invention, useful in determining if an individual has an age of onset marker I or an age of onset marker II, are allele-specific oligonucleotides. As used herein, the term allele-specific oligonucleotide (ASO) means an oligonucleotide that is able, under sufficiently stringent conditions, to hybridize specifically to one allele of a gene, or other locus, at a target region containing a PS while not hybridizing to the corresponding region in another allele(s). As understood by the skilled artisan, allele-specificity will depend upon a variety of readily optimized stringency conditions, including salt and formamide concentrations, as well as temperatures for both the hybridization and washing steps. Examples of hybridization and washing conditions typically used for ASO probes are found in Kogan et al., “Genetic Prediction of Hemophilia A” in PCR PROTOCOLS, A GUIDE TO METHODS AND APPLICATIONS, Academic Press, 1990, and Ruaño et al., Proc. Natl. Acad. Sci. USA 87:6296-300 (1990). Typically, an ASO will be perfectly complementary to one allele while containing a single mismatch for another allele.

Allele-specific oligonucleotides of the invention include ASO probes and ASO primers. ASO probes which usually provide good discrimination between different alleles are those in which a central position of the oligonucleotide probe aligns with the polymorphic site in the target region (e.g., approximately the 7^{th} or 8^{th} position in a 15mer, the 8^{th} or 9^{th} position in a 16mer, and the 10^{th} or 11^{th} position in a 20mer). An ASO primer of the invention has a 3′ terminal nucleotide, or preferably a 3′ penultimate nucleotide, that is complementary to only one of the nucleotide alleles of a particular SNP, thereby acting as a primer for polymerase-mediated extension only if that nucleotide allele is present at the PS in the sample being genotyped. ASO probes and primers hybridizing to either the coding or noncoding strand are contemplated by the invention. ASO probes and primers listed below use the appropriate nucleotide symbol (R=G or A, Y=T or C, M=A or C, K=G or T, S=G or C, and W=A or TJU; WIPO standard ST.25) at the position of the PS to represent that the ASO contains either of the two alternative allelic variants observed at that PS.

A preferred ASO probe for detecting the alleles at each of PS1, PS2, PS3, PS5, PS6, PS7, PS9, and PS10 is listed in Table 4. Additionally, detection of the alleles at each of PS1, PS2, PS3, PS5, PS6, PS7, PS9, and PS10 could be accomplished by utilization of the complement of these ASO probes.

A preferred ASO forward and reverse primer for detecting the alleles at each of PS1, PS2, PS3, PS5, PS6, PS7, PS9, and PS10 is listed in Table 4.

TABLE 4

Preferred ASOs for Detecting Alleles at PSs
in Haplotypes Comprising Preferred Embodiments
of Age of Onset Markers I and II¹

| | | |
|---|---|---|
| ASO Probe | ASO Forward | ASO Reverse |

| | | | | | | |
|---|---|---|---|---|---|---|
| | | SEQ | Primer | SEQ | Primer | SEQ |
| | | ID | | ID | | ID |
| PS | Sequence | NO. | Sequence | NO. | Sequence | NO. |

| | | | | | | |
|---|---|---|---|---|---|---|
| 1 | ATTGCTCWC | 2 | ACAGGGATTG | 11 | CGAGCTCCCC | 20 |
| | TCGGGG | | CTCWC | | GAGWG | |
| 2 | CTCACTCSG | 3 | GGATTGCTCA | 12 | GAGCCGAGCT | 21 |
| | GGAGCT | | CTCSG | | CCCSG | |
| 3 | CTTCCTTGTT | 4 | AAACCCCTTC | 13 | ACGCTGAGTT | 22 |
| | AACT | | CTTG | | AACAAGCA | |
| 3 | CTTCCTTGCT | 5 | AAACCCCTTC | 14 | ACGCTGAGTT | 23 |
| | TGTTAACT | | CTTGCTTG | | AACA | |
| 5 | GGTGCTGRG | 6 | CAGGTTGGTG | 15 | CCCTCCCAGG | 24 |
| | TGCCTG | | CTGRG | | CACYC | |
| 6 | GGACACCSG | 7 | TGGGAGGGAC | 16 | GCAGAGTGTA | 25 |
| | CCTACA | | ACCSG | | GGCSG | |
| 7 | TGTCGATSGT | 8 | TGGTTCTGTC | 17 | TACCTTCCAA | 26 |
| | CTTGG | | GATSG | | GACSA | |
| 9 | TGGAAGART | 9 | GAGATTTGGA | 18 | GTAGCCACCT | 27 |
| | GAAGGT | | AGART | | TCAYT | |
| 10 | AGAGGACST | 10 | CGGCTTAGAG | 19 | CGGAGCTCTC | 28 |
| | CTGAGA | | GACST | | AGASG | |

¹These ASO probes and primers include the appropriate nucleotide symbol, Y = T or C, R = G or A, M = A or C, S = G or C, and W = A or T/U (World Intellectual Property Organization Handbook on Industrial Property Information and Documentation IPO Standard ST.25 (1998), Appendix 2, Table 1), at the position of the PS to represent that the ASO contains one of the two alternative polymorphisms observed at that position.

Other oligonucleotides useful in practicing the invention hybridize to a target region located one to several nucleotides downstream of a PS in an age of onset marker. Such oligonucleotides are useful in polymerase-mediated primer-extension methods for detecting an allele at one of the PSs in the markers described herein and therefore such oligonucleotides are referred to herein as “primer-extension oligonucleotides.” In a preferred embodiment, the 3′-terminus of a primer-extension oligonucleotide is a deoxynucleotide complementary to the nucleotide located immediately adjacent to the PS. A particularly preferred forward and reverse primer-extension oligonucleotide for detecting the alleles at each of PS1, PS2, PS3, PS5, PS6, PS7, PS9, and PS10 is listed in Table 5. Termination mixes are chosen to terminate extension of the oligonucleotide at the PS of interest, or one base thereafter, depending on the alternative nucleotides present at the PS.

TABLE 5

Preferred Primer Extension Oligonucleotides
for Detecting Alleles at PSs in Haplotypes
Comprising Preferred Embodiments of
Age of Onset Markers I and II

| | Forward | | Reverse | |
|---|---|---|---|---|
| | Primer Extension | | Primer Extension | |
| PS | Sequence | SEQ ID NO. | Sequence | SEQ ID NO. |

| | | | | |
|---|---|---|---|---|
| 1 | GGGATTGCTC | 29 | GCTCCCCGAG | 37 |
| 2 | TTGCTCACTC | 30 | CCGAGCTCCC | 38 |
| 3 | CCCCTTCCTT | 31 | CTGAGTTAAC | 39 |
| 5 | GTTGGTGCTG | 32 | TCCCAGGCAC | 40 |
| 6 | GAGGGACACC | 33 | GAGTGTAGGC | 41 |
| 7 | TTCTGTCGAT | 34 | CTTCCAAGAC | 42 |
| 9 | ATTTGGAAGA | 35 | GCCACCTTCA | 43 |
| 10 | CTTAGAGGAC | 36 | AGCTCTCAGA | 44 |

In some embodiments, the oligonucleotides in a kit of the invention have different labels to allow probing of the identity of nucleotides or nucleotide pairs at two or more PSs simultaneously.

The oligonucleotides in a kit of the invention may also be immobilized on or synthesized on a solid surface such as a microchip, bead, or glass slide (see, e.g., WO 98/20020 and WO 98/20019). Such immobilized oligonucleotides may be used in a variety of polymorphism detection assays, including but not limited to probe hybridization and polymerase extension assays. Immobilized oligonucleotides useful in practicing the invention may comprise an ordered array of oligonucleotides designed to rapidly screen a nucleic acid sample for polymorphisms in multiple genes at the same time.

Kits of the invention may also contain other components such as hybridization buffer (e.g., where the oligonucleotides are to be used as allele-specific probes) or dideoxynucleotide triphosphates (ddNTPs; e.g., where the alleles at the polymorphic sites are to be detected by primer extension). In a preferred embodiment, the set of oligonucleotides consists of primer-extension oligonucleotides. The kit may also contain a polymerase and a reaction buffer optimized for primer-extension mediated by the polymerase. Preferred kits may also include detection reagents, such as biotin- or fluorescent-tagged oligonucleotides or ddNTPs and/or an enzyme-labeled antibody and one or more substrates that generate a detectable signal when acted on by the enzyme. It will be understood by the skilled artisan that the set of oligonucleotides and reagents for performing the genotyping or haplotyping assay will be provided in separate receptacles placed in the container if appropriate to preserve biological or chemical activity and enable proper use in the assay.

In a particularly preferred embodiment, each of the oligonucleotides and all other reagents in the kit have been quality tested for optimal performance in an assay for determining the alleles at a set of PSs comprising an age of onset marker I or age of onset marker II.

The invention provides a method for predicting the age of onset of AD in an individual at risk for developing AD. The method comprises determining whether the individual has an age of onset marker I or an age of onset marker II, and making an age of onset prediction based on the results of the determining step. The determination of the age of onset marker present in an individual can be made using one of the direct or indirect methods described herein. In some preferred embodiments, the determining step comprises identifying for one or both copies of the genomic locus present in the individual the identity of the nucleotide or nucleotide pair at the set of PSs comprising the selected age of onset marker. Alternatively, the determining step may comprise consulting a data repository that states the individual's copy number for the haplotypes comprising one of the age of onset markers I or age of onset markers II. The data repository may be the individual's medical records or a medical data card. In preferred embodiments, the individual is Caucasian.

According to Table 7 below, if the individual is determined to have an age of onset marker I, then the prediction is that the individual's age of onset of AD will be between 72.1 and 73.9, and if the individual is determined to have an age of onset marker II, then the prediction is that the individual's age of onset of AD will be between 68.3 and 72.2.

The invention further provides a method for delaying the onset of AD in an individual at risk for developing AD. The method comprises determining whether the individual has an age of onset marker I or an age of onset marker II, and making a treatment decision based upon the results of the determining step. In some embodiments, the determining step comprises identifying for one or both copies of the genomic locus present in the individual the identity of the nucleotide or nucleotide pair at the set of PSs comprising the selected haplotype. Alternatively, the determining step may comprise consulting a data repository that states the individual's copy number for a haplotype comprising an age of onset marker I or an age of onset marker II. The data repository may be the individual's medical records or a medical data card. In preferred embodiments, the individual is Caucasian.

If the individual is determined to have an age of onset marker I, the treatment decision is to prescribe to the individual a compound effective in delaying the onset of AD, wherein the compound is prescribed to the individual at an age below that of the lower confidence interval of the least square mean of age of onset for the age of onset marker I. If the individual is determined to have an age of onset marker II, the treatment decision is to prescribe to the individual at an age below that of the lower confidence interval of the least square mean of age of onset for the age of onset marker II. According to Table 7 below, the lower confidence interval of the least square mean of age of onset for an age of onset marker I is 72.1, and the lower confidence interval of the least square mean of age of onset for an age of onset marker II ranges from 68.3 to 68.4.

In other aspects, the invention provides an article of manufacture. In one embodiment, an article of manufacture comprises a pharmaceutical formulation and at least one indicium identifying a population for which the pharmaceutical formulation is indicated. The pharmaceutical formulation comprises, as at least one active ingredient, a compound effective in delaying the onset of AD in an individual at risk for developing AD. Additionally, the pharmaceutical formulation may be regulated and the indicium may comprise the approved label for the pharmaceutical formulation. The identified population is one that is at risk for developing AD, and is further partially or wholly defined by having an age of onset marker I or an age of onset marker II, wherein a trial population of individuals having an age of onset marker I exhibit a later age of onset of AD than a trial population of individuals having an age of onset marker II. The identified population preferably may be further defined as Caucasian. In addition to being at risk for developing AD, a population wholly defined by having an age of onset marker I or II is one for which there are no other factors which should be considered in identifying the population for which the pharmaceutical formulation is indicated. In contrast, a population that is partially defined by having an age of onset marker I or II is one for which other factors may be pertinent to identification of the population for which the pharmaceutical formulation is indicated. Examples of other such factors are age, weight, gender, disease state, possession of other genetic markers or biomarkers, or the like.

The pharmaceutical formulation may be formulated, in any way known in the art, for any mode of delivery (i.e., oral), and any mode of release (i.e., sustained release). In some embodiments, the pharmaceutical formulation is a tablet or capsule and the article may further comprise an additional indicium comprising the color or shape of the table or capsule. In other embodiments, the article may further comprise an additional indicium comprising a symbol stamped on the tablet or capsule, or a symbol or logo printed on the approved label.

In some embodiments of this article, the approved label may comprise a statement that the pharmaceutical formulation is indicated for delaying the onset of AD in an individual at risk for developing AD. In some embodiments, the approved label may further state the lower confidence interval of the least square mean of age of onset of AD for individuals having an age of onset marker I, and the lower confidence interval of the least square mean of age of onset of AD for individuals having an age of onset marker II.

An additional embodiment of the article of manufacture provided by the invention comprises packaging material and a pharmaceutical formulation contained within said packaging material. The pharmaceutical formulation comprises, as at least one active ingredient, a compound effective in delaying the onset of AD in an individual at risk for developing AD. Additionally, the packaging material may comprise a label stating that the pharmaceutical formulation is indicated for a population at risk for developing AD and which is partially or wholly defined by having an age of onset marker I or an age of onset marker II, and preferably further stating that a trial population of individuals having an age of onset marker I exhibit a later age of onset of AD than a trial population of individuals having an age of onset marker II. The indicated population preferably may be further defined as Caucasian.

Additionally, in other aspects of the invention, a method of manufacturing a drug product comprising, as at least one active ingredient, a compound effective in delaying the onset of AD in an individual at risk for developing AD is provided. The method comprises combining in a package a pharmaceutical formulation comprising the compound and a label that states that the formulation is indicated for delaying the onset of AD in a population at risk for developing AD and which is partially or wholly defined by having an age of onset marker I or an age of onset marker II, wherein a trial population having an age of onset marker I exhibits a later age of onset of AD than a trial population having an age of onset marker II. The indicated population may be identified on the pharmaceutical formulation, on the label or on the package by at least one indicium, such as a symbol or logo, color, or the like. The indicated population preferably may be further defined as Caucasian.

Detecting the presence of an age of onset marker I or an age of onset marker II in an individual is also useful in a method for seeking regulatory approval for marketing a pharmaceutical formulation for delaying the onset of AD in a population at risk for developing AD, wherein the population is partially or wholly defined by having an age of onset marker I or an age of onset marker II. The method comprises conducting at least one clinical trial which comprises administering the pharmaceutical formulation to first and second groups of individuals at risk for developing AD, and administering a placebo to third and fourth groups of individuals at risk for developing AD, wherein each individual in the first and third groups has an age of onset marker I, and each individual in the second and fourth groups has an age of onset marker II, demonstrating that the first group exhibits a later age of onset of AD than the third group, and demonstrating that the second group exhibits a later age of onset than the fourth group, and filing with a regulatory agency an application for marketing approval of the pharmaceutical formulation with a label stating that the pharmaceutical formulation is indicated for delaying the onset of AD in individuals at risk for developing AD. In preferred embodiments, the regulatory agency is the United States Food and Drug Administration (FDA) or the European Agency for the Evaluation of Medicinal Products (EMEA), or a future equivalent of these agencies.

The clinical trial may be conducted by recruiting individuals at risk for developing AD, determining whether they have an age of onset marker I or an age of onset marker II, and assigning them to the first and third groups if they have an age of onset marker I, and assigning them to the second and fourth groups if they have an age of onset marker II. The individuals in each of the first and second groups are preferably administered the same dose of the pharmaceutical formulation, and the individuals in each of the third and fourth groups are preferably administered the same does of the placebo.

The regulatory agency may be any person or group authorized by the government of a country anywhere in the world to control the marketing or distribution of drugs in that country. Preferably, the regulatory agency is authorized by the government of a major industrialized country, such as Australia, Canada, China, a member of the European Union, Japan, and the like. Most preferably the regulatory agency is authorized by the government of the United States and the type of application for approval that is filed will depend on the legal requirements set forth in the last enacted version of the Food, Drug and Cosmetic Act that are applicable for the pharmaceutical formulation and may also include other considerations such as the cost of making the regulatory filing and the marketing strategy for the composition.

For example, if the pharmaceutical formulation has previously been approved for the same cognitive function, then the application might be a paper NDA, a supplemental NDA or an abbreviated NDA, but the application would be a full NDA if the pharmaceutical formulation has never been approved before; with these terms having the meanings applied to them by those skilled in the pharmaceutical arts or as defined in the Drug Price Competition and Patent Term Restoration Act of 1984.

Additionally, in other aspects of the invention, there is provided a method for marketing a drug product comprising promoting to a target audience the use of a drug product for delaying the onset of AD in a population at risk for developing AD, wherein the population is partially or wholly defined by having an age of onset marker I or an age of onset marker II, wherein the drug product comprises a compound effective in delaying the onset of AD, and wherein a trial population of individuals having an age of onset marker I exhibit a later age of onset of AD than a trial population having an age of onset marker II. The target audience can be members of a group that is in position to influence prescription or purchase of the drug product. Such groups include physicians, pharmacists, insurance companies and health maintenance organizations, individuals at risk for developing AD, and government agencies such as those involved in providing or regulating medical insurance and those involved in regulating the marketing of drugs.

The promoting step can employ printed publications such as medical journals and consumer magazines, radio and television advertisements, and public presentations such as presentations at medical and scientific conferences. In a preferred embodiment, the drug product is approved for marketing to delay the onset of AD in the population, and the promoting step includes a statement that relates the approved drug product to its appearance, e.g., the color or shape of a tablet or capsule formulation, or some design stamped or embossed thereon.

Further, in performing any of the methods described herein which require information on the haplotype content of the individual (i.e., the haplotypes and haplotype copy number present in the individual for the polymorphic sites in haplotypes comprising an age of onset marker I or an age of onset marker II) or which require knowing if an age of onset marker I or an age of onset marker II is present in the individual, the individual's APOC1 haplotype content or age of onset marker may be determined by consulting a data repository such as the individual's patient records, a medical data card, a file (e.g., a flat ASCII file) accessible by a computer or other electronic or non-electronic media on which information about the individual's APOC1 haplotype content or age of onset marker can be stored. As used herein, a medical data card is a portable storage device such as a magnetic data card, a smart card, which has an on-board processing unit and which is sold by vendors such as Siemens of Munich Germany, or a flash-memory card. The medical data card may be, but does not have to be, credit-card sized so that it easily fits into pocketbooks, wallets and other such objects carried by the individual. The medical data card may be swiped through a device designed to access information stored on the data card. In an alternative embodiment, portable data storage devices other than data cards can be used. For example, a touch-memory device, such as the “i-button” produced by Dallas Semiconductor of Dallas, Tex. can store information about an individual's APOC1 haplotype content or age of onset marker, and this device can be incorporated into objects such as jewelry. The data storage device may be implemented so that it can wirelessly communicate with routing/intelligence devices through IEEE 802.112 wireless networking technology or through other methods well known to the skilled artisan. Further, as stated above, information about an individual's haplotype content or age of onset marker can also be stored in a file accessible by a computer; such files may be located on various media, including: a server, a client, a hard disk, a CD, a DVD, a personal digital assistant such as a Palm Pilot, a tape, a zip disk, the computer's internal ROM (read-only-memory) or the internet or worldwide web. Other media for the storage of files accessible by a computer will be obvious to one skilled in the art.

Any or all analytical and mathematical operations involved in practicing the methods of the present invention may be implemented by a computer. For example, the computer may execute a program that assigns APOC1 haplotype pairs and/or an age of onset marker I or an age of onset marker II to individuals based on genotype data inputted by a laboratory technician or treating physician. In addition, the computer may output the predicted change in cognitive function in age of onset to a galantamine following input of the individual's APOC1 haplotype content or age of onset marker, which was either determined by the computer program or input by the technician or physician. Data on which age of onset markers were detected in an individual may be stored as part of a relational database (e.g., an instance of an Oracle database or a set of ASCII flat files) containing other clinical and/or haplotype data for the individual. These data may be stored on the computer's hard drive or may, for example, be stored on a CD ROM or on one or more other storage devices accessible by the computer. For example, the data may be stored on one or more databases in communication with the computer via a network.

It is also contemplated that the above described methods and compositions of the invention may be utilized in combination with identifying genotype(s) and/or haplotype(s) for other genomic regions.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims that follow the examples.

### EXAMPLES

The Examples herein are meant to exemplify the various aspects of carrying out the invention and are not intended to limit the scope of the invention in any way. The Examples do not include detailed descriptions for conventional methods employed, such as in the synthesis of oligonucleotides or polymerase chain reaction. Such methods are well known to those skilled in the art and are described in numerous publications, for example, MOLECULAR CLONING: A LABORATORY MANUAL, 2^{nd} ed., supra.

### Example 1

This example illustrates the clinical and biochemical characterization of selected individuals in a cohort of 449 Caucasian patients diagnosed with AD, each of whom had previously participated in a clinical trial of galantamine.

### Example 2

Genomic DNA samples were isolated from blood samples obtained from each member of the cohort and variation within the APOC1 gene was examined by DNA sequencing through capillary electrophoresis. In brief, two regions of interest within the APOC1 gene were amplified by the polymerase chain reaction (PCR) using the primers listed below. The PCR products were then sequenced using dye-terminator sequencing and electrophoresed on an Applied Biosystems 3700 DNA Analyzer.

For each of the regions of interest, a genomic DNA sample was amplified using “tailed” PCR primers, each of which includes a universal sequence forming a noncomplementary tail attached to the 5′ end of each unique sequence in the PCR primer pairs. The universal tail sequence for the forward PCR primers comprises the sequence TGTAAAACGACGGCCAGT (SEQ ID NO:45) and the universal tail sequence for the reverse PCR primers comprises the sequence AGGAAACAGCTATGACCAT (SEQ ID NO:46). The nucleotide sequences of the forward and reverse primers for each region amplified are presented below in Table 6.

TABLE 6
APOC1 Amplification Primers

| | |
|---|---|
| Region 1 (Forward) | |
| AATCGATCACGACCCTCTCACG | (SEQ ID NO:47) |
| Region 1 (Reverse) | |
| CACTCAGCACCAACCTGAATCC | (SEQ ID NO:48) |
| Region 2 (Forward) | |
| GCTTTGGGGCTAAGGGAAGTGC | (SEQ ID NO:49) |
| Region 2 (Reverse) | |
| GGCAACCACGACCCAGAAGG | (SEQ ID NO:50) |

These primer pairs were used in PCR reactions containing genomic DNA isolated as described above. The PCR reactions were carried out in a 10 μl reaction volume under the following conditions:

Reaction Mixture

10× Titanium Polymerase reaction buffer (Clontech) (1 μl)

20 ng of human genomic DNA (2 μl)

10 mM dNTP (0.2 μl)

Titanium Polymerase enzyme mix (Clontech) (0.1 μl)

Forward Primer (25 μM) (0.12 μl)

Reverse Primer (25 μM) (0.12 μl)

Water (4.5 μl)

Amplification profile

| | | |
|---|---|---|
| 94° C.-two minutes | | one cycle |
| 97° C.-15 seconds | | 23 cycles, decreasing annealing (70° C.) |
| | {close oversize brace} | |
| 70° C.-45 seconds | | temperature by 0.7° C. per cycle |
| 72° C.-45 seconds | | |
| 97° C.-15 seconds | | |
| | {close oversize brace} | 22 cycles |
| 55° C.-45 seconds | | |
| 72° C.-45 seconds | | |

The PCR products were purified using a Whatman/Polyfiltronics 100 μl 384 well unifilter plate essentially according to the manufacturer's protocol. The purified DNA was eluted in 50 ll of distilled water. Sequencing reactions were set up using Applied Biosystems Big Dye Terminator chemistry essentially according to the manufacturer's protocol. The purified PCR products were sequenced in both directions using the appropriate universal ‘tail’ sequence as a primer. Reaction products were purified by isopropanol precipitation, and run on an Applied Biosystems 3700 DNA Analyzer.

Sequence information was analyzed for the presence of polymorphisms using SNP Scorer (Genaissance Pharmaceuticals, Inc., New Haven, Conn.) to view and analyze the results of the Polyphred program (Nickerson et al., Nucleic Acids Res. 14:2745-51 (1997)).

### Example 3

This example illustrates the deduction of haplotypes from the APOC1 genotyping data generated in Example 2.

Haplotypes were estimated from the unphased genotypes using a computer-implemented algorithm for assigning haplotypes to unrelated individuals in a population sample, essentially as described in WO 01/80156 (Genaissance Pharmaceuticals, Inc., New Haven, Conn.). In this method, haplotypes are assigned directly from individuals who are homozygous at all sites or heterozygous at no more than one of the variable sites. This list of haplotypes is then used to deconvolute the unphased genotypes in the remaining (multiply heterozygous) individuals.

A quality control analysis was performed on the deduced haplotypes, which included analysis of the frequencies of the haplotypes and individual SNPs therein for compliance with principles of Hardy-Weinberg equilibrium.

### Example 4

This example illustrates analysis of the APOC1 haplotypes in Table 1 for association with individuals' age of onset of Alzheimer's Disease.

The statistical analyses compared age of onset of AD in individuals with one copy or two copies vs. zero copies, or zero copies or one copy vs. two copies (within an individual's genome) of a particular allele, using a logistic regression analysis on two-degrees of freedom to associate age of onset of AD with a particular haplotype. The following covariates were also included: gender, family history, and smoking.

For the results obtained on the analyses, adjustments were made for multiple comparisons, using a permutation test (MULTIVARIATE PERMUTATION TESTS: WITH APPLICATIONS IN BIOSTATISTICS, Pesarin, John Wiley and Sons, New York, 2001). In this test, a haplotype's data for each observation were kept constant, while all the remaining variables (outcome and covariates) were randomly permuted so that covariates always stayed with the same outcome. The permutation model was fitted for each of the several haplotypes, and the lowest p-value was kept. In total, 1000 permutations were done. Sixty-eight APOC1 haplotypes of at least one polymorphism were identified that show a correlation with an individual's age of onset of AD. These APOC1 haplotypes are shown above in Table 1, and the unadjusted (“Raw”) and adjusted (“Perm.”) p-values for these 68 haplotypes are shown below in Table 7.

TABLE 7
APOC1 Haplotypes Having Association with Age of Onset of Alzheimer's Disease

| | | | | | Lower | Upper |
|---|---|---|---|---|---|---|
| | | | | | Confidence | Confidence |
| | | | Subject | Least Square | Interval of Least | Interval of Least |
| | | | Count for | Mean of Age | Square Mean of | Square Mean of |
| | | | Haplotype (# | of Onset (# of | Age of Onset (# | Age of Onset (# |
| Haplotype | Perm. p | Raw p | of copies) | copies) | of copies) | of copies) |
| (1) | 0.033 | 0.012832 | 68 (0) | 70.3 (0) | 68.4 (0) | 72.2 (0) |
| | | | 395 (1 or 2) | 73.0 (1 or 2) | 72.1 (1 or 2) | 73.9 (1 or 2) |
| (2) | 0.033 | 0.012832 | 68 (0) | 70.3 (0) | 68.4 (0) | 72.2 (0) |
| | | | 395 (1 or 2) | 73.0 (1 or 2) | 72.1 (1 or 2) | 73.9 (1 or 2) |
| (3) | 0.033 | 0.012832 | 68 (0) | 70.3 (0) | 68.4 (0) | 72.2 (0) |
| | | | 395 (1 or 2) | 73.0 (1 or 2) | 72.1 (1 or 2) | 73.9 (1 or 2) |
| (4) | 0.033 | 0.012832 | 68 (0) | 70.3 (0) | 68.4 (0) | 72.2 (0) |
| | | | 395 (1 or 2) | 73.0 (1 or 2) | 72.1 (1 or 2) | 73.9 (1 or 2) |
| (5) | 0.033 | 0.012832 | 68 (0) | 70.3 (0) | 68.4 (0) | 72.2 (0) |
| | | | 395 (1 or 2) | 73.0 (1 or 2) | 72.1 (1 or 2) | 73.9 (1 or 2) |
| (6) | 0.033 | 0.012832 | 68 (0) | 70.3 (0) | 68.4 (0) | 72.2 (0) |
| | | | 395 (1 or 2) | 73.0 (1 or 2) | 72.1 (1 or 2) | 73.9 (1 or 2) |
| (7) | 0.033 | 0.012832 | 68 (0) | 70.3 (0) | 68.4 (0) | 72.2 (0) |
| | | | 395 (1 or 2) | 73.0 (1 or 2) | 72.1 (1 or 2) | 73.9 (1 or 2) |
| (8) | 0.033 | 0.012832 | 68 (0) | 70.3 (0) | 68.4 (0) | 72.2 (0) |
| | | | 395 (1 or 2) | 73.0 (1 or 2) | 72.1 (1 or 2) | 73.9 (1 or 2) |
| (9) | 0.033 | 0.012832 | 68 (0) | 70.3 (0) | 68.4 (0) | 72.2 (0) |
| | | | 395 (1 or 2) | 73.0 (1 or 2) | 72.1 (1 or 2) | 73.9 (1 or 2) |
| (10) | 0.033 | 0.012832 | 68 (0) | 70.3 (0) | 68.4 (0) | 72.2 (0) |
| | | | 395 (1 or 2) | 73.0 (1 or 2) | 72.1 (1 or 2) | 73.9 (1 or 2) |
| (11) | 0.033 | 0.012832 | 68 (0) | 70.3 (0) | 68.4 (0) | 72.2 (0) |
| | | | 395 (1 or 2) | 73.0 (1 or 2) | 72.1 (1 or 2) | 73.9 (1 or 2) |
| (12) | 0.033 | 0.012832 | 68 (0) | 70.3 (0) | 68.4 (0) | 72.2 (0) |
| | | | 395 (1 or 2) | 73.0 (1 or 2) | 72.1 (1 or 2) | 73.9 (1 or 2) |
| (13) | 0.033 | 0.012832 | 68 (0) | 70.3 (0) | 68.4 (0) | 72.2 (0) |
| | | | 395 (1 or 2) | 73.0 (1 or 2) | 72.1 (1 or 2) | 73.9 (1 or 2) |
| (14) | 0.033 | 0.012832 | 68 (0) | 70.3 (0) | 68.4 (0) | 72.2 (0) |
| | | | 395 (1 or 2) | 73.0 (1 or 2) | 72.1 (1 or 2) | 73.9 (1 or 2) |
| (15) | 0.033 | 0.012832 | 68 (0) | 70.3 (0) | 68.4 (0) | 72.2 (0) |
| | | | 395 (1 or 2) | 73.0 (1 or 2) | 72.1 (1 or 2) | 73.9 (1 or 2) |
| (16) | 0.033 | 0.012832 | 68 (0) | 70.3 (0) | 68.4 (0) | 72.2 (0) |
| | | | 395 (1 or 2) | 73.0 (1 or 2) | 72.1 (1 or 2) | 73.9 (1 or 2) |
| (17) | 0.033 | 0.012832 | 68 (0) | 70.3 (0) | 68.4 (0) | 72.2 (0) |
| | | | 395 (1 or 2) | 73.0 (1 or 2) | 72.1 (1 or 2) | 73.9 (1 or 2) |
| (18) | 0.033 | 0.012832 | 68 (0) | 70.3 (0) | 68.4 (0) | 72.2 (0) |
| | | | 395 (1 or 2) | 73.0 (1 or 2) | 72.1 (1 or 2) | 73.9 (1 or 2) |
| (19) | 0.033 | 0.012832 | 68 (0) | 70.3 (0) | 68.4 (0) | 72.2 (0) |
| | | | 395 (1 or 2) | 73.0 (1 or 2) | 72.1 (1 or 2) | 73.9 (1 or 2) |
| (20) | 0.033 | 0.012832 | 68 (0) | 70.3 (0) | 68.4 (0) | 72.2 (0) |
| | | | 395 (1 or 2) | 73.0 (1 or 2) | 72.1 (1 or 2) | 73.9 (1 or 2) |
| (21) | 0.033 | 0.012832 | 68 (0) | 70.3 (0) | 68.4 (0) | 72.2 (0) |
| | | | 395 (1 or 2) | 73.0 (1 or 2) | 72.1 (1 or 2) | 73.9 (1 or 2) |
| (22) | 0.033 | 0.012832 | 68 (0) | 70.3 (0) | 68.4 (0) | 72.2 (0) |
| | | | 395 (1 or 2) | 73.0 (1 or 2) | 72.1 (1 or 2) | 73.9 (1 or 2) |
| (23) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (24) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (25) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (26) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (27) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (28) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (29) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (30) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (31) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (32) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (33) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (34) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (35) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (36) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (37) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (38) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (39) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (40) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (41) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (42) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (43) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (44) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (45) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (46) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (47) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (48) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (49) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (50) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (51) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (52) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (53) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (54) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (55) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (56) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (57) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (58) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (59) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (60) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (61) | 0.034 | 0.013206 | 63 (0) | 70.3 (0) | 68.3 (0) | 72.2 (0) |
| | | | 293 (1 or 2) | 73.0 1 or 2) | 72.1 (1 or 2) | 73.9 (1 or 2) |
| (62) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (63) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (64) | 0.034 | 0.013206 | 63 (0) | 70.3 (0) | 68.3 (0) | 72.2 (0) |
| | | | 293 (1 or 2) | 73.0 (1 or 2) | 72.1 (1 or 2) | 73.9 (1 or 2) |
| (65) | 0.034 | 0.013206 | 293 (0 or 1) | 73.0 (0 or 1) | 72.1 (0 or 1) | 73.9 (0 or 1) |
| | | | 63(2) | 70.3 (2) | 68.3 (2) | 72.2 (2) |
| (66) | 0.034 | 0.013206 | 293 (0 or 1) | 73.0 (0 or 1) | 72.1 (0 or 1) | 73.9 (0 or 1) |
| | | | 63(2) | 70.3 (2) | 68.3 (2) | 72.2 (2) |
| (67) | 0.034 | 0.013206 | 293 (0 or 1) | 73.0 (0 or 1) | 72.1 (0 or 1) | 73.9 (0 or 1) |
| | | | 63(2) | 70.3 (2) | 68.3 (2) | 72.2 (2) |
| (68) | 0.034 | 0.013206 | 293 (0 or 1) | 73.0 (0 or 1) | 72.1 (0 or 1) | 73.9 (0 or 1) |
| | | | 63(2) | 70.3 (2) | 68.3 (2) | 72.2 (2) |

As seen in Table 7, each of the 68 haplotypes shows a correlation with an individual's age of onset of AD. When p-values were adjusted for multiple comparisons, haplotypes (1)-(22) showed the strongest correlation. The Least Square Mean of Age of Onset column indicates the average age of onset of AD in individuals, in this cohort, having (a) one copy or two copies, or zero copies, or (b) zero copies or one copy, or two copies of a particular haplotype.

In view of the above, it will be seen that the several advantages of the invention are achieved and other advantageous results attained. As various changes could be made in the above methods and compositions without departing from the scope of the invention, it is intended that all matter contained in the above description and shown in the accompanying drawings shall be interpreted as illustrative and not in a limiting sense.

All references cited in this specification, including patents and patent applications, are hereby incorporated in their entirety by reference. The discussion of references herein is intended merely to summarize the assertions made by their authors and no admission is made that any reference constitutes prior art. Applicants reserve the right to challenge the accuracy and pertinence of the cited references.

1. A method for determining whether an individual has an age of onset marker I or an age of onset marker II, the method comprising:
determining whether the individual has (a) one copy or two copies, or zero copies of any of (i) haplotypes (1)-(64) in Table 1, (ii) a linked haplotype for any of haplotypes (1)-(64) in Table 1, and (iii) a substitute haplotype for any of haplotypes (1)-(64) in Table 1, or (b) zero copies or one copy, or two copies of any of (i) haplotypes (65)-(68) in Table 1, (ii) a linked haplotype for any of haplotypes (65)-(68) in Table 1, and (iii) a substitute haplotype for any of haplotypes (65)-(68) in Table 1, wherein the polymorphic sites (PSs) in haplotypes (1)-(68) in Table 1 correspond to the following nucleotide positions in SEQ ID NO:1: PS1, 106; PS2, 110; PS3, 172; PS5, 536; PS6, 555; PS7, 731; PS9, 787; PS10, 827, wherein the individual has an age of onset marker I if the individual has (a) one copy or two copies of any of (i) haplotypes (1)-(64) in Table 1, (ii) a linked haplotype for any of haplotypes (1)-(64) in Table 1, and (iii) a substitute haplotype for any of haplotypes (1)-(64) in Table 1, or (b) zero copies or one copy of any of (i) haplotypes (65)-(68) in Table 1, (ii) a linked haplotype for any of haplotypes (65)-(68) in Table 1, and (iii) a substitute haplotype for any of haplotypes (65)-(68) in Table 1, and the individual has an age of onset marker II if the individual has (a) zero copies of any of (i) haplotypes (1)-(64) in Table 1, (ii) a linked haplotype for any of haplotypes (1)-(64) in Table 1, and (iii) a substitute haplotype for any of haplotypes (1)-(64) in Table 1, or (b) two copies of any of (i) haplotypes (65)-(68) in Table 1, (ii) a linked haplotype for any of haplotypes (65)-(68) in Table 1, and (iii) a substitute haplotype for any of haplotypes (65)-(68) in Table 1.

2. The method of claim 1, wherein the determining step comprises obtaining the individual's genotype for each PS in the set of PSs comprising any of (a) haplotypes (1)-(68) in Table 1, (b) a linked haplotype for any of of haplotypes (1)-(68) in Table 1, and (c) a substitute haplotype for any of haplotypes (1)-(68) in Table 1, and using the results of the obtaining step to identify the pair of haplotypes for the set of PSs.

3. The method of claim 2, wherein the individual's genotype for the set of PSs is obtained by any of (a) a primer extension assay; (b) an allele-specific PCR assay; (c) a nucleic acid amplification assay; (d) a hybridization assay; (e) a mismatch-detection assay; (f) an enzymatic nucleic acid cleavage assay; and (g) a sequencing assay.

4. The method of claim 1; wherein the determining step comprises consulting a data repository that provides information on the individual's copy number for any of (a) haplotypes (1)-(68) in Table 1, (b) a linked haplotype for any of haplotypes (1)-(68) in Table 1, and (c) a substitute haplotype for any of haplotypes (1)-(68) in Table 1.

5. The method of claim 4, wherein the data repository is the individual's medical records or a medical data card.

6. The method of claim 1, wherein the method comprises determining whether an individual has one copy or two copies, or zero copies of any of (a) haplotype (23) in Table 1, (b) a linked haplotype for haplotype (23) in Table 1, and (c) a substitute haplotype for haplotype (23) in Table 1.

7. The method of claim 6, wherein the method comprises determining whether an individual has one copy or two copies, or zero copies of haplotype (23) in Table 1.

8. The method of claim 1, wherein the linkage disequilibrium between the linked haplotype and at least one of haplotypes (1) -(68) in Table 1 has a delta squared value selected from the group consisting of at least 0.75, at least 0.80, at least 0.85, at least 0.90, at least 0.95, and 1.0.

9. The method of claim 8, wherein the linked haplotype is for haplotype (23) in Table 1 and the linkage disequilibrium between the linked haplotype and haplotype (23) in Table 1 has a delta squared value of at least 0.95.

10. The method of claim 1, wherein the linkage disequilibrium between the allele at a substituting PS in the substitute haplotype and the allele at a substituted PS in any of haplotypes (1)-(68) in Table 1 has a delta squared value selected from the group consisting of at least 0.75, least 0.80, at least 0.85, at least 0.90, at least 0.95, and 1.0.

11. The method of claim 10, wherein the linkage disequilibrium between the allele at a substituting PS and the allele at a substituted PS in haplotype (23) in Table 1 has a delta squared value of at least 0.95.

12. The method of claim 1, wherein the individual is Caucasian.

13. A method for assigning an individual to a first age of onset marker group or a second age of onset marker group, the method comprising:
determining whether the individual has (a) one copy or two copies, or zero copies of any of (i) haplotypes (1)-(64) in Table 1, (ii) a linked haplotype for any of haplotypes (1)-(64) in Table 1, and (iii) a substitute haplotype for any of haplotypes (1)-(64) in Table 1, or (b) zero copies or one copy, or two copies of any of (i) haplotypes (65)-(68) in Table 1, (ii) a linked haplotype for any of haplotypes (65)-(68) in Table 1, and (iii) a substitute haplotype for any of haplotypes (65)-(68) in Table 1, wherein the polymorphic sites (PSs) in haplotypes (1)-(68) in Table 1 correspond to the following nucleotide positions in SEQ ID NO:1: PS1, 106; PS2, 110; PS3, 172; PS5, 536; PS6, 555; PS7, 731; PS9, 787; PS 10, 827; and
assigning the individual to the first age of onset marker group if the individual has (a) one copy or two copies of any of (i) haplotypes (1)-(64) in Table 1, (ii) a linked haplotype for any of haplotypes (1)-(64) in Table 1, and (iii) a substitute haplotype for any of haplotypes (1)-(64) in Table 1, or (b) zero copies one copy of any of (i) haplotypes (65)-(68) in Table 1, (ii) a linked haplotype for any of haplotypes (65)-(68) in Table 1, and (iii) a substitute haplotype for any of haplotypes (65)-(68) in Table 1, and assigning the individual to the second age of onset marker group if the individual has (a) zero copies of any of (i) haplotypes (1)-(64) in Table 1, (ii) a linked haplotype for any of haplotypes (1)-(64) in Table 1, and (iii) a substitute haplotype for any of haplotypes (1)-(64) in Table 1, or (b) two copies of any of (i) haplotypes (65)-(68) in Table 1, (ii) a linked haplotype for any of haplotypes (65)-(68) in Table 1, and (iii) a substitute haplotype for any of haplotypes (65)-(68) in Table 1.

14. The method of claim 13, wherein the determining step comprises obtaining the individual's genotype for each PS in the set of PSs comprising any of (a) haplotypes (1)-(68) in Table 1, (b) a linked haplotype for any of of haplotypes (1)-(68) in Table 1, and (c) a substitute haplotype for any of haplotypes (1)-(68) in Table 1, and using the results of the obtaining step to identify the pair of haplotypes for the set of PSs.

15. The method of claim 14, wherein the individual's genotype for the set of PSs is obtained by any of (a) a primer extension assay; (b) an allele-specific PCR assay; (c) a nucleic acid amplification assay; (d) a hybridization assay; (e) a mismatch-detection assay; (f) an enzymatic nucleic acid cleavage assay; and (g) a sequencing assay.

16. The method of claim 13, wherein the determining step comprises consulting a data repository that provides information on the individual's copy number for any of (a) haplotypes (1)-(68) in Table 1, (b) a linked haplotype for any of haplotypes (1)-(68) in Table 1, and (c) a substitute haplotype for any of haplotypes (1)-(68) in Table 1.

17. The method of claim 16, wherein the data repository is the individual's medical records or a medical data card.

18. The method of claim 13, wherein the method comprises:
determining whether the individual has one copy or two copies, or zero copies of any of (a) haplotype (23) in Table 1, (b) a linked haplotype for haplotype (23) in Table 1, and (c) a substitute haplotype for haplotype (23) in Table 1; and
assigning the individual to the first age of onset marker group if the individual has one copy or two copies of any of (a) haplotype (23) in Table 1, (b) a linked haplotype for haplotype (23) in Table 1, and (c) a substitute haplotype for haplotype (23) in Table 1, and assigning the individual to the second age of onset marker group if the individual has zero copies of any of (a) haplotype (23) in Table 1, (b) a linked haplotype for haplotype (23) in Table 1, and (c) a substitute haplotype for haplotype (23) in Table 1.

19. The method of claim 18, wherein the method comprises:
determining whether the individual has one copy or two copies, or zero copies of haplotype (23) in Table 1; and
assigning the individual to the first age of onset marker group if the individual has one copy or two copies of haplotype (23) in Table 1, and assigning the individual to the second age of onset marker group if the individual has zero copies of haplotype (23) in Table 1.

20. The method of claim 13, wherein the individual is Caucasian.

21. The method of claim 13, wherein the linkage disequilibrium between the linked haplotype and at least one of haplotypes (1)-(68) in Table 1 has a delta squared value selected from the group consisting of at least 0.75, at least 0.80, at least 0.85, at least 0.90, at least 0.95, and 1.0.

22. The method of claim 21, wherein the linked haplotype is for haplotype (23) in Table 1 and the linkage disequilibrium between the linked haplotype and haplotype (23) in Table 1 has a delta squared value of at least 0.95.

23. The method of claim 13, wherein the linkage disequilibrium between the allele at a substituting PS in the substitute haplotype and the allele at a substituted PS in any of haplotypes (1)-(68) in Table 1 has a delta squared value selected from the group consisting of at least 0.75, least 0.80, at least 0.85, at least 0.90, at least 0.95, and 1.0.

24. The method of claim 23, wherein the linkage disequilibrium between the allele at a substituting PS and the allele at a substituted PS in haplotype (23) in Table 1 has a delta squared value of at least 0.95.

25. A kit for determining whether an individual has an age of onset marker I or an age of onset marker II, the kit comprising a set-of one or more oligonucleotides designed for identifying at least one of the alleles at each polymorphic site (PS) in a set of one or more PSs, wherein the set of one or more PSs comprises: (a) PS3 and PS7; (b) PS3, PS5, PS7, and PS9; (c) PS1, PS3, PS7, and PS9; (d) PS3, PS6, PS7, and PS9; (e) PS2, PS3, PS7, and PS9; (f) PS3, PS7, PS9, and PS10; (g) PS2, PS3, PS5, and PS7; (h) PS3, PS5, and PS7; (i) PS1, PS3, and PS7; (j) PS2, PS3, and PS7; (k) PS3, PS6, and PS7; (l) PS3, PS7, and PS10; (m) PS1, PS3; PS5, and PS7; (n) PS3, PS5, PS6, and PS7; (o) PS1, PS3, PS6, and PS7; (p) PS1, PS2, PS3, and PS7; (q) PS2, PS3, PS6, and PS7; (r) PS3, PS5, PS7, and PS10; (s) PS1, PS3, PS7, and PS10; (t) PS3, PS7, and PS9; (u) PS3, PS6, PS7, and PS10; (v) PS2, PS3, PS7, and PS10; (w) PS3; (x) PS3, PS5, PS6, and PS9; (y) PS3, PS5, and PS10; (z) PS2, PS3, PS9, and PS10; (aa) PS1, PS3, PS6, and PS10; (bb) PS1, PS3, and PS5; (cc) PS1, PS2, PS3, and PS9; (dd) PS3, PS6, and PS9; (ee) PS3 and PS10; (ff) PS2, PS3, PS5, and PS9; (gg) PS1 and PS3; (hh) PS3, PS5, PS6, and PS10; (ii) PS1, PS3, PS5, and PS6; (jj) PS1, PS2, PS3, and PS10; (kk) PS3 and PS5; (ll) PS3, PS6, and PS10; (mm) PS2, PS3, and PS9; (nn) PS1, PS3, and PS6; (oo) PS2, PS3, PS5, and PS10; (pp) PS1, PS2, PS3, and PS5; (qq) PS3, PS5, and PS6; (rr) PS1, PS3, PS9, and PS10; (ss) PS2, PS3, PS6, and PS9; (tt) PS2, PS3, and PS10; (uu) PS1, PS2, and PS3; (vv) PS3 and PS6; (ww) PS3, PS5, PS9, and PS10; (xx) PS1, PS3, PS5, and PS9; (yy) PS2, PS3, and PS5; (zz) PS2, PS3, PS6, and PS10; (aaa) PS3, PS9, and PS10; (bbb) PS1, PS2, PS3, and PS6; (ccc) PS1, PS3, and PS9; (ddd) PS2 and PS3; (eee) PS2, PS3, PS5, and PS6; (fff) PS1, PS3, PS5, and PS10; (ggg) PS3, PS6, PS9, and PS10; (hhh) PS3, PS5, and PS9; (iii) PS1, PS3, PS6, and PS9; (jjj) PS1, PS3, and PS10; (kkk) PS2, PS3, and PS6; (lll) PS3 and PS9; (mmm) PS3; (nnn) PS3 and PS7; (ooo) PS1 and PS3; (ppp) PS1, PS3, and PS7; (qqq) a set of one or more PSs in a linked haplotype for any of haplotypes (1)-(68) in Table 1, or (rrr) a set of one or more PSs in a substitute haplotype for any of haplotypes (1)-(68) in Table 1, wherein the enumerated PSs in sets (a)-(ppp) correspond to the following nucleotide positions in SEQ ID NO: 1: PS1, 106; PS2, 110; PS3, 172; PS5, 536; PS6, 555; PS7, 731; PS9, 787; PS10, 827.

26. The kit of claim 25, wherein the kit comprises a set of one or more oligonucleotides designed for identifying at least one of the alleles at each PS in a set of one or more PSs, wherein the set of one or more PSs is any of: (a) PS3 and PS7; (b) PS3, PS5, PS7, and PS9; (c) PS1, PS3, PS7, and PS9; (d) PS3, PS6, PS7, and PS9; (e) PS2, PS3, PS7, and PS9; (f) PS3, PS7, PS9, and PS10; (g) PS2, PS3, PS5, and PS7; (h) PS3, PS5, and PS7; (i) PS1, PS3, and PS7; () PS2, PS3, and PS7; (k) PS3, PS6, and PS7; (l) PS3, PS7, and PS10; (m) PS1, PS3, PS5, and PS7; (n) PS3, PS5, PS6, and PS7; (o) PS1, PS3, PS6, and PS7; (p) PS1, PS2, PS3, and PS7; (q) PS2, PS3, PS6, and PS7; (r) PS3, PS5, PS7, and PS10; (s) PS1, PS3, PS7, and PS10; (t) PS3, PS7, and PS9; (u) PS3, PS6, PS7, and PS10; (v) PS2, PS3, PS7, and PS10; (w) PS3; (x) PS3, PS5, PS6, and PS9; (y) PS3, PS5, and PS10; (z) PS2, PS3, PS9, and PS10; (aa) PS1, PS3, PS6, and PS 10; (bb) PS1, PS3, and PS5; (cc) PS1, PS2, PS3, and PS9; (dd) PS3, PS6, and PS9; (ee) PS3 and PS10; (ff) PS2, PS3, PS5, and PS9; (gg) PS1 and PS3; (hh) PS3, PS5, PS6, and PS10; (ii) PS1, PS3, PS5, and PS6; (jj) PS1, PS2, PS3, and PS10; (kk) PS3 and PS5; (ll) PS3, PS6, and PS10; (mm) PS2, PS3, and PS9; (nn) PS1, PS3, and PS6; (oo) PS2, PS3, PS5, and PS10; (pp) PS1, PS2, PS3, and PS5; (qq) PS3, PS5, and PS6; (rr) PS1, PS3, PS9, and PS10; (ss) PS2, PS3, PS6, and PS9; (tt) PS2, PS3, and PS10; (uu) PS1, PS2, and PS3; (vv) PS3 and PS6; (ww) PS3, PS5, PS9, and PS10; (xx) PS1, PS3, PS5, and PS9; (yy) PS2, PS3, and PS5; (zz) PS2, PS3, PS6, and PS10; (aaa) PS3, PS9, and PS10; (bbb) PS1, PS2, PS3, and PS6; (ccc) PS1, PS3, and PS9; (ddd) PS2 and PS3; (eee) PS2, PS3, PS5, and PS6; (fff) PS1, PS3, PS5, and PS10; (ggg) PS3, PS6, PS9, and PS10; (hhh) PS3, PS5, and PS9; (iii) PS1, PS3, PS6, and PS9; (jjj) PS1, PS3, and PS10; (kkk) PS2, PS3, and PS6; (lll) PS3 and PS9; (mmm) PS3; (nnn) PS3 and PS7; (ooo) PS1 and PS3; (ppp) PS1, PS3, and PS7; (qqq) a set of one or more PSs in a linked haplotype for any of haplotypes (1)-(68) in Table 1, and (rrr) a set of one or more PSs in a substitute haplotype for any of haplotypes (1)-(68) in Table 1, wherein the enumerated PSs in sets (a)-(ppp) correspond to the following nucleotide positions in SEQ ID NO:1: PS1, 106; PS2, 110; PS3, 172; PS5, 536; PS6, 555; PS7, 731;PS9, 787;PS10, 827.

27. The kit of claim 25, wherein the set of one or more oligonucleotides is designed for identifying both alleles at each PS in the set of one or more PSs.

28. The kit of claim 25, wherein the set of one or more PSs is (w), (qqq), or (rrr), wherein if the set is (qqq), then the linked haplotype is a linked haplotype for haplotype (23) in Table 1, and wherein if the set is (rrr), then the substitute haplotype is a substitute haplotype for haplotype (23) in Table 1.

29. The kit of claim 28, wherein the set of one or more PSs is (w).

30. The kit of claim 25, wherein the individual is Caucasian.

31. The kit of claim 25, which further comprises a manual with instructions for (a) performing one or more reactions on a human nucleic acid sample to identify the allele or alleles present in the individual at each PS in the set of one or more PSs, and (b) determining if the individual has an age of onset marker I or an age of onset marker II based on the identified allele or alleles.

32. The kit of claim 25, wherein the linkage disequilibrium between the linked haplotype and at least one of haplotypes (1)-(68) in Table 1 has a delta squared value selected from the group consisting of at least 0.75, at least 0.80, at least 0.85, at least 0.90, at least 0.95, and 1.0.

33. The kit of claim 25, wherein the set of one or more PSs is (w) or (qqq), wherein if the set is (qqq), then the linked haplotype is a linked haplotype for haplotype (23) in Table 1 and the linkage disequilibrium between the linked haplotype and haplotype (23) in Table 1 has a delta squared value of at least 0.95.

34. The kit of claim 25, wherein the linkage disequilibrium between the allele at a substituting PS in the substitute haplotype and the allele at a substituted PS in any of haplotypes (1)-(68) in Table 1 has a delta squared value selected from the group consisting of at least 0.75, at least 0.80, at least 0.85, at least 0.90, at least 0.95, and 1.0.

35. The kit of claim 25, wherein the set of one or more PSs is (w) or (rrr), wherein if the set is (rrr), then the substitute haplotype is a substitute haplotype for haplotype (23) in Table 1 and the linkage disequilibrium between the allele at a substituting PS in the substitute haplotype and the allele at a substituted PS in haplotype (23) in Table 1 has a delta squared value of at least 0.95.

36. The kit of claim 25, wherein at least one oligonucleotide in the set of one or more oligonucleotides is an allele-specific oligonucleotide (ASO) probe comprising a nucleotide sequence, wherein the sequence is any of SEQ ID NOS:2-10 and their complements.

37. The kit of claim 36, wherein the, set of one or more PSs is (w) and the at least one oligonucleotide in the set of one or more oligonucleotides is a first ASO probe and a second ASO probe, wherein the first ASO probe comprises a nucleotide sequence, wherein the sequence is SEQ ID NO:4 or its complement, and wherein the second ASO probe comprises a nucleotide sequence, wherein the sequence is SEQ ID NO:5 or its complement.

38. The kit of claim 25, wherein at least one oligonucleotide in the set of one or more oligonucleotides is a primer-extension oligonucleotide comprising a nucleotide sequence, wherein the sequence is any of SEQ ID NOS:11-44.

39. The kit of claim 38, wherein the set of one or more PSs is (e) and the at least one oligonucleotide in the set of one or more oligonucleotides is a first primer-extension oligonucleotide, wherein the first primer extension oligonucleotide comprises a nucleotide sequence, wherein the sequence is any of SEQ ID NO:31 and SEQ ID NO:39.

40. A method for delaying the onset of Alzheimer's Disease (AD) in an individual at risk for developing AD, the method comprising:
determining whether the individual has an age of onset marker I or an age of onset marker II; and
choosing a treatment for the individual based upon the results of the determining step.

41. The method of claim 40, wherein if the individual has an age of onset marker I, then the chosen treatment is prescribing to the individual a compound effective in delaying the onset of AD, at an age below that of the lower confidence interval of the least square mean of age of onset for the age of onset marker I, and wherein if the individual has an age of onset marker II, then the chosen treatment is prescribing to the individual a compound effective in delaying the onset of AD, at an age below that of the lower confidence interval of the least square mean of age of onset for the age of onset marker II.

42. The method of claim 40, wherein the determining step comprises consulting a data repository that states whether the individual has an age of onset marker I or an age of onset marker II.

43. The method of claim 42, wherein said data repository is the individual's medical records or a medical data card.

44. A method for predicting the age of onset of Alzheimer's Disease (AD) in an individual at risk for developing AD, the method comprising:
determining whether the individual has an age of onset marker I or an age of onset marker II; and
making an age of onset prediction based on the results of the determining step.

45. The method of claim 44, wherein if the individual is determined to have an age of onset marker I, then the prediction is that the individual will develop AD between 72.1 and 73.9, and if the individual is determined to have an age of onset marker II, then the prediction is that the individual will develop AD between 68.3 and 72.2.

46. The method of claim 44, wherein the determining step comprises consulting a data repository that states whether the individual has an age of onset marker I or an age of onset marker II.

47. The method of claim 46, wherein the data repository is the individual's medical records or a medical data card.

48. An article of manufacture, comprising a pharmaceutical formulation and at least one indicium identifying a population for whom the pharmaceutical formulation is indicated, wherein the pharmaceutical formulation comprises, as at least one active ingredient, a compound effective in delaying the onset of Alzheimer's Disease (AD), and wherein the identified population is at risk for developing AD and is partially or wholly defined by having an age of onset marker I or an age of onset marker II.

49. The article of manufacture of claim 48, wherein marketing of the pharmaceutical formulation is regulated and the indicium comprises the approved label for the pharmaceutical formulation.

50. The article of manufacture of claim 48, wherein the compound is present in the pharmaceutical formulation at an amount effective to delay the onset of AD.

51. The article of manufacture of claim 48, wherein the age of onset marker I is one copy or two copies of any of (a) haplotype (23) in Table 1, (b) a linked haplotype for haplotype (23) in Table 1, and (c) a substitute haplotype for haplotype (23) in Table 1, and the age of onset marker II is zero copies of any of (a) haplotype (23) in Table 1, (b) a linked haplotype for haplotype (23) in Table 1, and (c) a substitute haplotype for haplotype (23) in Table 1.

52. The article of manufacture of claim 51, wherein the linkage disequilibrium between the linked haplotype and haplotype (23) in Table 1 has a delta squared value selected from the group consisting of at least 0.75, at least 0.80, at least 0.85, at least 0.90, at least 0.95, and 1.0.

53. The article of manufacture of claim 52, wherein the delta squared value is at least 0.95.

54. The article of manufacture of claim 51, wherein the linkage disequilibrium between the allele at a substituting PS in the substitute haplotype and the allele at a substituted PS in haplotype (23) in Table 1 has a delta squared value selected from the group consisting of at least 0.75, least 0.80, at least 0.85, at least 0.90, at least 0.95, and 1.0.

55. The article of manufacture of claim 54, wherein the delta squared value is at least 0.95.

56. The article of manufacture of claim 48, further comprising an additional indicium identifying the population.

57. The article of manufacture of claim 56, wherein the pharmaceutical formulation is a tablet or capsule and the additional indicium comprises the color or shape of the tablet or capsule.

58. The article of manufacture of claim 56, wherein the pharmaceutical formulation is a tablet or capsule and the additional indicium comprises a symbol stamped on the tablet or capsule.

59. The article of manufacture of claim 48, wherein the identified population is further defined as being Caucasian.

60. An article of manufacture, comprising packaging material and a pharmaceutical formulation contained within the packaging material, wherein the pharmaceutical formulation comprises, as at least one active ingredient, a compound effective in delaying the onset of Alzheimer's Disease (AD), and wherein the packaging material comprises a label which states that the pharmaceutical formulation is indicated for a population at risk for developing AD that is partially or wholly defined by having a age of onset marker I or age of onset marker II.

61. The article of manufacture of claim 60 wherein the age of onset marker I is one copy or two copies of any of (a) haplotype (23) in Table 1, (b) a linked haplotype for haplotype (23) in Table 1, and (c) a substitute haplotype for haplotype (23) in Table 1, and the age of onset marker II is zero copies of any of (a) haplotype (23) in Table 1, (b) a linked haplotype for haplotype (23) in Table 1, and (c) a substitute haplotype for haplotype (23) in Table 1.

62. The article of manufacture of claim 61, wherein the age of onset marker I is one copy or two copies of haplotype (23) in Table 1, and the age of onset marker II is zero copies of haplotype (23) in Table 1.

63. The article of manufacture of claim 61, wherein the linkage disequilibrium between the linked haplotype and haplotype (23) in Table 1 has a delta squared value selected from the group consisting of at least 0.75, least 0.80, at least 0.85, at least 0.90, at least 0.95, and 1.0.

64. The article of manufacture of claim 63, wherein the delta squared value is at least 0.95.

65. The article of manufacture of claim 61, wherein the linkage disequilibrium between the allele at a substituting PS in the substitute haplotype and the allele at a substituted PS in haplotype (23) in Table 1 has a delta squared value selected from the group consisting of at least 0.75, least 0.80, at least 0.85, at least 0.90, at least 0.95, and 1.0.

66. The article of manufacture of claim 65, wherein the delta squared value is at least 0.95.

67. A method for manufacturing a drug product, the method comprising combining in a package a pharmaceutical formulation comprising, as at least one active ingredient, a compound effective in delaying the onset of Alzheimer's Disease (AD), and a label which states that the pharmaceutical formulation is indicated for a population at risk for developing AD that is partially or wholly defined by having an age of onset marker I or an age of onset marker II.

68. The method of claim 67, wherein the age of onset marker I is one copy or two copies of any of (a) haplotype (23) in Table 1, (b) a linked haplotype for haplotype (23) in Table 1, and (c) a substitute haplotype for haplotype (23) in Table 1, and the age of onset marker II is zero copies of any of (a) haplotype (23) in Table 1, (b) a linked haplotype for haplotype (23) in Table 1, and (c) a substitute haplotype for haplotype (23) in Table 1.

69. The method of claim 68, wherein the linkage disequilibrium between the linked haplotype and haplotype (23) in Table 1 has a delta squared value selected from the group consisting of at least 0.75, at least 0.90, at least 0.95, and 1.0.

70. The method of claim 69, wherein the delta squared value is at least 0.95.

71. The method of claim 68, wherein the linkage disequilibrium between the allele at a substituting PS in the substitute haplotype and the allele at a substituted PS in haplotype (23) in Table 1 has a delta squared value selected from the group consisting of at least 0.75, least 0.80, at least 0.85, at least 0.90, at least 0.95, and 1.0.

72. The method of claim 71, wherein the delta squared value is at least 0.95.

73. The method of claim 67, wherein the label further states that the indicated population is further defined as being Caucasian.


SEQUENCE LISTING (50 sequences)

SEQ ID NO: 1 - DNA, 5375 bp, Homo sapiens
  misc_feature (106)..(106): n is ′a′ or ′t′
gcatgcagcccccagtcacgcatcccctgcttgttcaatcgatcacgaccctctcacgtgcacccacttagagttgtgagcccttaaaaggaacagggattgctcnctcngggagctcggctcttgagacaggaatcttgcccattccccgaacgaataaaccccttccttcgttaactcagcgtctgaggaattttgtctgcggctcctcctgctacattctgagtgggggaaagggactaaggtggtctgaggaccccacagagtcaggaagattgagaggtgagagtgctgaacggggaggggctttggggctaagggaagtgcccgggaccccacctgaccccaacgctcacgggacaggggcagaggagaaaaacntgggtggacagagggaggcaggcggtcaggggaaggctcaggaggagggagatcaacatcaacctgccccgcccctccccagcctgataaaggtcctgcgggcaggacaggacctcccaaccaagccctccagcaaggattcaggttggtgctgngtgcctgggagggacaccngcctacactctgcaagaaactcaaaaagggagatgaggggatcgtgggagggaggtagggagggaggagggtgccactgatcccctgaacccctgcctctgcctccagagtgcccctccggcctcgccatgaggctcttcctgtcgctcccggtcctggtggtggttctgtcgatngtcttggaaggtaaaagtgggatgggagaattggggagtttngagatttggaagantgaaggtggctacaggcctggggtcccggcttagaggacntctgagagctccggggccccttctgggtcgtggttgcctcatcgtggtcgggtgggtctccaggttctcccaggctcagtcccgcaggcgccaaatctgcgcaggagagcactagcaaccgatgacgtattgaggcccacacctctgggattggctgtcctgcttcgacagccttgaaagtgggtaagctgggtggggggctctgggagaggtcagtgctgagtaaggcaattcccagcagcttgagccccaccaggtcactccagtattcctccccattctttttttttttttttttttttctcttgagacggagtctcgctctgtcgccgaggctggagtgcagtggcgcgatctcggctcactgcaagctccgcctccctggttcacgccattctcctgcctcagcaggactacaggcgcccgcctccgcgcccggctaattttttgtattttcagtagagacagggtttcaccgtggtctcgatctcctgactttgtgatccgcctgcctcgacctcccaaagtgctgggattacaggcgtgagccaccgcgtccggccattcctccccattctaaccacatgatccccaaggatctctatccatcccggtatcccaacctaagggggttccaataacaaatttttggccgggcagggtggctcatgcctgtaatcccagcactttgggaggccgaggcgggcagatcacttgaggtcaggagttcaaaccagccaggccaacatggtggaaacttcgtctctagctaaaaatacaaaaaaattaggccaggtgtggaggcacgcgcctgtaggcccagctactcgggaggctgaggcaggagaatcacttgaacccgggaggcggaggttgcagtgagccgagatcataccactgcactccagcctggctgacacagcaagactccgtctcaaaacaaaacaaaacaaaaataggctgggtgtggtggtgcacacctgtaatcccagctacttgggaggctgaggcaggagaactgcttgaacccgggaggtggtggttgcagtaggccgagatcatgccactgcactccagcttgggctacagagcaagactccatctccaaaaaaaaaaaaaaaaaaacaaattttgaacccctgcccatcttcctggcaggcccagccccagcccaggggaccccagacgtctccagtgccttggataagctgaaggagtttggaaacacactggaggacaaggctcgggaactcatcagccgcatcaaacagagtgaactttctgccaagatgcggttagaacccttcccagggcacgggagagctggggtgtgtttttgggtggagccctggcagatggtccaagatgaacagattgaaaaaaaaacaagtcctggagaggctgacaacatccctctggtcacacagctagatctcaaggtgctcagacttcaaggacagtttccctgactcccatccaggccatattttaaaagatggtcttgggctgggcacggtggctcatgcttgcaatcccagcacttagggaggccgaggtgggctgattgcctgaggtcaggagttcgagaccagtctgaccaacatcggtgaaatcttagtctctactaaaaatacaaaaaaattacggcaggcatggtggcgtgcactgtaatcccagctagtcgggaggctgaggcaggggaattgcttgaaccaggaaggtgggagttacagtgagccaacattgtgccagcctgggtgacagaaggagactctgtctcaaaaaaaaaaaaaaaaaaaaaaaaaaacaagatggtcttgcccaggaatggtggctaacacctgtaattccagcattatgggaggctgagatgggaggattgcttgagcccaggagttcgagaccagcctgaccaacatggcgagatcctgtctccatttaaaaaaaaaaaaaaaaagatggttttgtgaggtaatgaaaatgaaggccccaagcttggccagacctgggtccccaggctggagtagcaccccttcctgtgtgatcttgacagaggggcattactgtgagcctcagtttcctctcctataaactggtggttctacagggaagtaaaggagcaggcctacagggtgtctggtacatgtagatgctcagtatatcattaaaccaccttcccctttgcaagttagagagtcatttgttctttaaaaaatattttactgagcatctgctaagtgctggaaaactctttcaatgtggggaataaaacagtgaagaactgccgagcacggtggctcacacctgtaaccccaccactttggaaggccgaggtgggtggatcacttgaggtcaggagtgcgagaaccccgtccctaatagaaatgcaaaaaaaattagctgggcatggtggcccatgcctgtagtcccagctccttgggagggctgaggcggagaggattgcttgagcccaggagatctaggctgcagtgcgccatgtttgtgccactgcattccagcctgggtaacagaatgagaccctgtctcaacaaaaaaagaaaagaaaagagaagaaaagagaaaagaaagacagggagggagggaggaaggaagggagggagggagggaaaatagagccaggcataaacttagaaagatcgtttggaggccaggcacaatggctcacacctgtaatcccagcactttgggaggccaaggcaagcagatcaactgaggtcaggagttcgagaccagcctaacatggagaaaacccctgtctctactaaaaaaaaatacaaaaaaattagccggggcgtggtgcattcctgtagtcctagctactcgggagcctgaggcaggagaatcacttgaacccgggaggcggaggttgcagtgagccgagatcatgccactgcactccagcctgggcgacaaggcgagactccatgccaaaaaagaaaaaaaactcctggcgcggtggctcacgccagtaatcccagcactgtgggaggctgagcaggcggatcacgaggtcaggagttcgagactagcctgctcaacataatgaaaccctctctgtactaaaaatacaaaaattagctgggtgtggtggcaggcacctgtagtcccagctactcgggaggctgaggcaggagaatggcttgaacctgggaggcagaggttgcagtgagccgagacagtgccattgcactccagtccaggtgacagagcgaaactccatctcaaaaaaaaaaggaaggcattggtagcaagagatggcaggccttgaaagccaggccagggtgaagtgtttctttttttttttttttttttttctttttaaatttttttttttgagacggagtctcgctctgtcacccaggctggattgcagtggcctgatctcggctcactgcaagttccgcctcccgggttcatgccattctcctgcctcaccctcccgagtagctgggactacaggcacctgccaccaggccagctaattttttgtattcttagtagaatgtagaatttacttagtagaattttttgtattcttagccagcatggtctcgatctcctgacctgggtgatccacccgcctcggcctcccaaagtgctgggattacaggcgtgagccacggcgcccggccttattttttctttttgagatgtacccagactggagtacagtggtgcgatctcggcttactggaacctccacctcccgggttcaggcaattctcctgcctcagcctcatgagtacttggaactacaggtgtgtgacaccacacatggtattttttgtatttttagtgaagatgacatttcaccatgttgcccaggttggtctcgaactcctgacctcaagtgatcagcctacctcggcctcccaaagtgttgggattacaggcgtgagccaaatgcccagccaagggtaaagtgtttagacttaaagtgctttggtccatctgggaaactgaggcagagaagttggcccacccagcccagcggtcctcctaatcccacagacagtggggatggagattctgcaaggggaagaggtgggagtcaggtagcaggcagaatttggacagcctgggaggtagctgcacacagtgacccccttccttattcctccccacagggagtggttttcagagacatttcagaaagtgaaggagaaactcaagattgactcatgaggacctgaagggtgacatcccaggaggggcctctgaaatttcccacaccccagcgcctgtgctgaggactccctccatgtggccccaggtgccaccaataaaaatcctacagaaaattctctcctgagtgcttctttactctggggaaggggctgcgggagagggtaggggcttccagagagggcagggtctgcagccactgtggaaaaacagtatggggtttcctcaaaacattaaagatagaactctcaaatgatccttcaatcccacttctgggtatttattcaaaagaattgaaatcaggaccttgaagagatacctgccctcccatgttcactgcag

SEQ ID NO: 2 - DNA, 15 bp, Artificial
  ASO Probe for Detecting Alleles at PSs in Haplotypes Comprising Preferred Embodiments of Age of Onset Markers I and II
attgctcwctcgggg

SEQ ID NO: 3 - DNA, 15 bp, Artificial
  ASO Probe for Detecting Alleles at PSs in Haplotypes Comprising Preferred Embodiments of Age of Onset Markers I and II
ctcactcsgggagct

SEQ ID NO: 4 - DNA, 14 bp, Artificial
  ASO Probe for Detecting Alleles at PSs in Haplotypes Comprising Preferred Embodiments of Age of Onset Markers I and II
cttccttgttaact

SEQ ID NO: 5 - DNA, 18 bp, Artificial
  ASO Probe for Detecting Alleles at PSs in Haplotypes Comprising Preferred Embodiments of Age of Onset Markers I and II
cttccttgcttgttaact

SEQ ID NO: 6 - DNA, 15 bp, Artificial
  ASO Probe for Detecting Alleles at PSs in Haplotypes Comprising Preferred Embodiments of Age of Onset Markers I and II
ggtgctgrgtgcctg

SEQ ID NO: 7 - DNA, 15 bp, Artificial
  ASO Probe for Detecting Alleles at PSs in Haplotypes Comprising Preferred Embodiments of Age of Onset Markers I and II
ggacaccsgcctaca

SEQ ID NO: 8 - DNA, 15 bp, Artificial
  ASO Probe for Detecting Alleles at PSs in Haplotypes Comprising Preferred Embodiments of Age of Onset Markers I and II
tgtcgatsgtcttgg

SEQ ID NO: 9 - DNA, 15 bp, Artificial
  ASO Probe for Detecting Alleles at PSs in Haplotypes Comprising Preferred Embodiments of Age of Onset Markers I and II
tggaagartgaaggt

SEQ ID NO: 10 - DNA, 15 bp, Artificial
  ASO Probe for Detecting Alleles at PSs in Haplotypes Comprising Preferred Embodiments of Age of Onset Markers I and II
agaggacstctgaga

SEQ ID NO: 11 - DNA, 15 bp, Artificial
  ASO Forward Primer for Detecting Alleles at PSs in Haplotypes Comprising Preferred Embodiments of Age of Onset Markers I and II
acagggattgctcwc

SEQ ID NO: 12 - DNA, 15 bp, Artificial
  ASO Forward Primer for Detecting Alleles at PSs in Haplotypes Comprising Preferred Embodiments of Age of Onset Markers I and II
ggattgctcactcsg

SEQ ID NO: 13 - DNA, 14 bp, Artificial
  ASO Forward Primer for Detecting Alleles at PSs in Haplotypes Comprising Preferred Embodiments of Age of Onset Markers I and II
aaaccccttccttg

SEQ ID NO: 14 - DNA, 18 bp, Artificial
  ASO Forward Primer for Detecting Alleles at PSs in Haplotypes Comprising Preferred Embodiments of Age of Onset Markers I and II
aaaccccttccttgcttg

SEQ ID NO: 15 - DNA, 15 bp, Artificial
  ASO Forward Primer for Detecting Alleles at PSs in Haplotypes Comprising Preferred Embodiments of Age of Onset Markers I and II
caggttggtgctgrg

SEQ ID NO: 16 - DNA, 15 bp, Artificial
  ASO Forward Primer for Detecting Alleles at PSs in Haplotypes Comprising Preferred Embodiments of Age of Onset Markers I and II
tgggagggacaccsg

SEQ ID NO: 17 - DNA, 15 bp, Artificial
  ASO Forward Primer for Detecting Alleles at PSs in Haplotypes Comprising Preferred Embodiments of Age of Onset Markers I and II
tggttctgtcgatsg

SEQ ID NO: 18 - DNA, 15 bp, Artificial
  ASO Forward Primer for Detecting Alleles at PSs in Haplotypes Comprising Preferred Embodiments of Age of Onset Markers I and II
gagatttggaagart

SEQ ID NO: 19 - DNA, 15 bp, Artificial
  ASO Forward Primer for Detecting Alleles at PSs in Haplotypes Comprising Preferred Embodiments of Age of Onset Markers I and II
cggcttagaggacst

SEQ ID NO: 20 - DNA, 15 bp, Artificial
  ASO Reverse Primer for Detecting Alleles at PSs in Haplotypes Comprising Preferred Embodiments of Age of Onset Markers I and II
cgagctccccgagwg

SEQ ID NO: 21 - DNA, 15 bp, Artificial
  ASO Reverse Primer for Detecting Alleles at PSs in Haplotypes Comprising Preferred Embodiments of Age of Onset Markers I and II
gagccgagctcccsg

SEQ ID NO: 22 - DNA, 18 bp, Artificial
  ASO Reverse Primer for Detecting Alleles at PSs in Haplotypes Comprising Preferred Embodiments of Age of Onset Markers I and II
acgctgagttaacaagca

SEQ ID NO: 23 - DNA, 14 bp, Artificial
  ASO Reverse Primer for Detecting Alleles at PSs in Haplotypes Comprising Preferred Embodiments of Age of Onset Markers I and II
acgctgagttaaca

SEQ ID NO: 24 - DNA, 15 bp, Artificial
  ASO Reverse Primer for Detecting Alleles at PSs in Haplotypes Comprising Preferred Embodiments of Age of Onset Markers I and II
ccctcccaggcacyc

SEQ ID NO: 25 - DNA, 15 bp, Artificial
  ASO Reverse Primer for Detecting Alleles at PSs in Haplotypes Comprising Preferred Embodiments of Age of Onset Markers I and II
gcagagtgtaggcsg

SEQ ID NO: 26 - DNA, 15 bp, Artificial
  ASO Reverse Primer for Detecting Alleles at PSs in Haplotypes Comprising Preferred Embodiments of Age of Onset Markers I and II
taccttccaagacsa

SEQ ID NO: 27 - DNA, 15 bp, Artificial
  ASO Reverse Primer for Detecting Alleles at PSs in Haplotypes Comprising Preferred Embodiments of Age of Onset Markers I and II
gtagccaccttcayt

SEQ ID NO: 28 - DNA, 15 bp, Artificial
  ASO Reverse Primer for Detecting Alleles at PSs in Haplotypes Comprising Preferred Embodiments of Age of Onset Markers I and II
cggagctctcagasg

SEQ ID NO: 29 - DNA, 10 bp, Artificial
  Forward Primer Extension Oligonucleotide for Detecting Alleles at PSs in Haplotypes Comprising Preferred Embodiments of Age of Onset Markers I and II
gggattgctc

SEQ ID NO: 30 - DNA, 10 bp, Artificial
  Forward Primer Extension Oligonucleotide for Detecting Alleles at PSs in Haplotypes Comprising Preferred Embodiments of Age of Onset Markers I and II
ttgctcactc

SEQ ID NO: 31 - DNA, 10 bp, Artificial
  Forward Primer Extension Oligonucleotide for Detecting Alleles at PSs in Haplotypes Comprising Preferred Embodiments of Age of Onset Markers I and II
ccccttcctt

SEQ ID NO: 32 - DNA, 10 bp, Artificial
  Forward Primer Extension Oligonucleotide for Detecting Alleles at PSs in Haplotypes Comprising Preferred Embodiments of Age of Onset Markers I and II
gttggtgctg

SEQ ID NO: 33 - DNA, 10 bp, Artificial
  Forward Primer Extension Oligonucleotide for Detecting Alleles at PSs in Haplotypes Comprising Preferred Embodiments of Age of Onset Markers I and II
gagggacacc

SEQ ID NO: 34 - DNA, 10 bp, Artificial
  Forward Primer Extension Oligonucleotide for Detecting Alleles at PSs in Haplotypes Comprising Preferred Embodiments of Age of Onset Markers I and II
ttctgtcgat

SEQ ID NO: 35 - DNA, 10 bp, Artificial
  Forward Primer Extension Oligonucleotide for Detecting Alleles at PSs in Haplotypes Comprising Preferred Embodiments of Age of Onset Markers I and II
atttggaaga

SEQ ID NO: 36 - DNA, 10 bp, Artificial
  Forward Primer Extension Oligonucleotide for Detecting Alleles at PSs in Haplotypes Comprising Preferred Embodiments of Age of Onset Markers I and II
cttagaggac

SEQ ID NO: 37 - DNA, 10 bp, Artificial
  Reverse Primer Extension Oligonucleotide for Detecting Alleles at PSs in Haplotypes Comprising Preferred Embodiments of Age of Onset Markers I and II
gctccccgag

SEQ ID NO: 38 - DNA, 10 bp, Artificial
  Reverse Primer Extension Oligonucleotide for Detecting Alleles at PSs in Haplotypes Comprising Preferred Embodiments of Age of Onset Markers I and II
ccgagctccc

SEQ ID NO: 39 - DNA, 10 bp, Artificial
  Reverse Primer Extension Oligonucleotide for Detecting Alleles at PSs in Haplotypes Comprising Preferred Embodiments of Age of Onset Markers I and II
ctgagttaac

SEQ ID NO: 40 - DNA, 10 bp, Artificial
  Reverse Primer Extension Oligonucleotide for Detecting Alleles at PSs in Haplotypes Comprising Preferred Embodiments of Age of Onset Markers I and II
gagtgtaggc

SEQ ID NO: 41 - DNA, 10 bp, Artificial
  Reverse Primer Extension Oligonucleotide for Detecting Alleles at PSs in Haplotypes Comprising Preferred Embodiments of Age of Onset Markers I and II
gagtgtaggc

SEQ ID NO: 42 - DNA, 10 bp, Artificial
  Reverse Primer Extension Oligonucleotide for Detecting Alleles at PSs in Haplotypes Comprising Preferred Embodiments of Age of Onset Markers I and II
cttccaagac

SEQ ID NO: 43 - DNA, 10 bp, Artificial
  Reverse Primer Extension Oligonucleotide for Detecting Alleles at PSs in Haplotypes Comprising Preferred Embodiments of Age of Onset Markers I and II
gccaccttca

SEQ ID NO: 44 - DNA, 10 bp, Artificial
  Reverse Primer Extension Oligonucleotide for Detecting Alleles at PSs in Haplotypes Comprising Preferred Embodiments of Age of Onset Markers I and II
agctctcaga

SEQ ID NO: 45 - DNA, 18 bp, Artificial
  The universal tail sequence for the forward PCR primers
tgtaaaacgacggccagt

SEQ ID NO: 46 - DNA, 19 bp, Artificial
  The universal tail sequence for the reverse PCR primers
aggaaacagctatgaccat

SEQ ID NO: 47 - DNA, 22 bp, Artificial
  APOC1 Amplification Primer
aatcgatcacgaccctctcacg

SEQ ID NO: 48 - DNA, 22 bp, Artificial
  APOC1 Amplification Primer
cactcagcaccaacctgaatcc

SEQ ID NO: 49 - DNA, 22 bp, Artificial
  APOC1 Amplification Primer
gctttggggctaagggaagtgc

SEQ ID NO: 50 - DNA, 20 bp, Artificial
  APOC1 Amplification Primer
ggcaaccacgacccagaagg